(19) United States
(12) Patent Application Publication
(10) Pub. No.: US 2002/0019000 A1
(43) Pub. Date: Feb. 14, 2002

(54) Polynucleotides coexpressed with matrix-remodeling genes

(76) Inventors: Michael G. Walker, Sunnyvale, CA (US); Wayne Volkmuth, Calabasas, CA (US); Tod M. Klingler, San Carlos, CA (US)

Correspondence Address: LEGAL DEPARTMENT, INCYTE GENOMICS, INC., 3160 PORTER DRIVE, PALO ALTO, CA, 94304, US

(21) Appl. No.: 09/818,143

(22) Filed: Mar. 26, 2001

Related U.S. Application Data
(63) Continuation-in-part of application No. 09/169,289, filed on Oct. 9, 1998.

(51) Int. Cl. C12Q 1/68; C07H 21/04

(52) U.S. Cl. 435/6; 536/23.1

(57) ABSTRACT

The invention provides compositions, polynucleotides and proteins that coexpress with known matrix-remodeling genes. The invention also provides expression vectors and host cells, and ligands and antibodies which specifically bind the proteins. The invention also relates to the use of these biomolecules in diagnosis, prognosis, prevention, treatment, and evaluation of therapies for diseases associated with matrix-remodeling.

This application is a continuation-in-part of U.S. Ser. No. 09/169,289, filed Oct. 9, 1998.

### FIELD OF THE INVENTION

The invention relates to novel polynucleotides and their encoded proteins which were identified by their coexpression with known matrix-remodeling genes. The invention also relates to the use of these biomolecules in diagnosis, prognosis, prevention, treatment, and evaluation of therapies for diseases, particularly diseases associated with matrix-remodeling such as angiogenesis, arthritis, atherosclerosis, cancers, cardiomyopathy, diabetic necrosis, fibrosis, and ulceration.

### BACKGROUND OF THE INVENTION

Matrix-remodeling is associated with the construction, destruction, and reorganization of extracellular matrix components and is essential in normal cellular functions and also in many disease processes. These disease processes include angiogenesis, arthritis, atherosclerosis, cancers, cardiomyopathy, diabetic necrosis, fibrosis, and ulceration (Alexander and Werb (1991) In: Cell Biology of Extracellular Matrix, Plenum Press, New York N.Y., pp. 255-302; Schuppan et al. (1993) In: Extracellular Matrix, Marcel Dekker, New York N.Y., pp. 201-254; Zvibel and Kraft (1993) In: Extracellular Matrix, Marcel Dekker, New York N.Y., pp. 559-580; Shanahan et al. (1994) J Clin Invest 93:2393-402; Kielty and Shuttleworth (1995) Int J Biochem Cell Biol 27:747-60; Bitar and Labbad (1996) J Surg Res 61:113-9; Dourado et al. (1996) Osteoarthritis Cartilage 4:187-96; Grant et al. (1996) Regul Pept 67:137-44; Gunja-Smith et al. (1996) Am J Pathol 148:1639-48; Alcolado et al. (1997) Clin Sci 92:103-12; Cs-Szabo et al. (1997) Arthritis Rheum 40:1037-45; Hayward and Brock (1997) Hum Mutat 10:415-23; Ledda et al. (1997) J Invest Dermatol 108:210-4; Hayashido et al. (1998) Int J Cancer 75:654-8; Ito et al. (1998) Kidney Int 53:853-61; and Nelson et al. (1998) Cancer Res 58:232-6).

Many genes that participate in and regulate matrix-remodeling are known, but many remain to be identified. Identification of currently unknown polynucleotides and their encoded proteins will provide new diagnostic and therapeutic targets. In addition, these newly discovered biomolecules will provide new opportunities for therapeutic tissue engineering—the use of drugs or biologicals to direct the creation of new tissues such as skin, pancreas, or liver that can replace tissues lost to disease or trauma.

The present invention provides new compositions, polynucleotides, and proteins that are useful for diagnosis, prognosis, treatment, and evaluation of therapies for diseases associated with matrix-remodeling. We have implemented a method for analyzing gene expression patterns and have identified 20 novel matrix-remodeling polynucleotides and their encoded protein by their coexpression with known matrix-remodeling genes.

### SUMMARY OF THE INVENTION

The invention provides for a composition comprising purified polynucleotides that are coexpressed with one or more known matrix-remodeling genes in a plurality of biological samples. Preferably, the known matrix-remodeling gene is selected from the group consisting of osteonectin (BM-40), chondroitin/dermatan sulfate proteoglycans (C/DSPG), collagen I, II, II, and IV, connective tissue growth factor (CTGF), fibrillin, fibronectins, fibronectin receptor (fibr-r), fibulin 1, heparan sulfate proteoglycans (HSPG), extracellular matrix protein (hevin), insulin-like growth factor 1 (IGF 1), insulin-like growth factor binding protein (IGFBP), laminin, lumican, matrix Gla protein (MGP), matrix metalloproteases (MMP), and tissue inhibitors of matrix metalloproteinase 1, 2, and 3 (TIMP 1, 2, and 3). A composition comprising a plurality of polynucleotides having the nucleic acid sequences of SEQ ID NOs:1-13 or the complements thereof.

The invention also provides a composition comprising a polynucleotide and a labeling moiety. The invention further provides a method of using a composition to screen a plurality of molecules to identify at least one ligand which specifically binds a polynucleotide of the composition, the method comprises combining the composition with molecules under conditions to allow specific binding; and detecting specific binding, thereby identifying a ligand which specifically binds the polynucleotide. In one aspect of the method, the molecules to be screened are selected from DNA molecules, RNA molecules, peptide nucleic acids, mimetics, and proteins. The invention still further provides a method for using a composition to detect gene expression in a sample containing nucleic acids, the method comprises hybridizing the composition to the nucleic acids under conditions for formation of one or more hybridization complexes; and detecting hybridization complex formation, wherein complex formation indicates gene expression in the sample. In one aspect of the method, the sample is derived from arteries, cancerous cells of any tissue or organ, cartilage, heart, lungs, pancreas, synovium or synovial fluid, and veins. In another aspect of the method, gene expression indicates the presence of angiogenesis, arthritis, atherosclerosis, cancers, cardiomyopathy, diabetic necrosis, fibrosis, and ulceration.

The invention provides an isolated polynucleotide comprising a nucleic acid sequence selected from SEQ ID NOs:1-20 or the complement thereof. The invention also provides a method of using a polynucleotide to purify a ligand, the method comprises combining the polynucleotide with a sample under conditions to allow specific binding; recovering the bound polynucleotide; and separating the ligand from the bound polynucleotide, thereby obtaining purified ligand. In one aspect of the method, \the polynucleotide is attached to a substrate. In another aspect of the method, the molecules to be screened are selected from DNA molecules, RNA molecules, peptide nucleic acids, mimetics, and proteins.

The method provides a vector comprising a polynucleotide selected from SEQ ID NOs:1-20. The invention also provides a host cell containing the vector. The invention further provides a method for using a host cell to produce a protein, the method comprises culturing the host cell under conditions for expression of the protein; and recovering the protein from cell culture.

The method provides a purified protein encoded by one of the polynucleotides of the invention. The invention also provides a composition comprising the protein and a pharmaceutical carrier. The invention further provides a method for using a protein to screen a plurality of molecules to identify at least one ligand which specifically binds the protein, the method comprises combining the protein with the plurality of molecules under conditions to allow specific binding; and detecting specific binding, thereby identifying a ligand which specifically binds the protein. In one aspect of the method, the plurality of molecules is selected from DNA molecules, RNA molecules, peptide nucleic acids, mimetics, proteins, agonists, antagonists, and antibodies. The invention still further provides a method of using a protein to purify a ligand from a sample, the method comprises combining the protein with a sample under conditions to allow specific binding; recovering the bound protein; and separating the ligand from the bound protein, thereby obtaining purified ligand.

### BRIEF DESCRIPTION OF THE SEQUENCE LISTING AND FIGURES

The Sequence Listing provides exemplary matrix-remodeling-associated polynucleotides and their encoded proteins including the nucleic acid sequences, SEQ ID NOs:1-20, and amino acid sequences, SEQ ID NOs:21-23. Each sequence is identified by a sequence identification number (SEQ ID NO) and by the Incyte Clone number in which the biomolecule was first identified.

FIGS. 1A, 1B, 1C, 1D, 1E, 1F, 1G, and 1H show the protein of SEQ ID NO:21 encoded by the polynucleotide of SEQ ID NO:2. The translation was produced using MACDNASIS PRO software (Hitachi Software Engineering, South San Francisco Calif.).

FIGS. 2A, 2B, 2C, and 2D show the protein of SEQ ID NO:22 encoded by the polynucleotide of SEQ ID NO:6. The translation was produced using MACDNASIS PRO software (Hitachi Software Engineering).

FIGS. 3A, 3B, 3C, 3D, 3E, 3F, and 3G show the protein of SEQ ID NO:23 encoded by the polynucleotide of SEQ ID NO:11. The translation was produced using MACDNASIS PRO software (Hitachi Software Engineering).

FIG. 4 shows the categories of tissues in which SEQ ID NO:3 is expressed. It serves as an example of the expression profile produced using the LIFESEQ Gold database (Incyte Genomics, Palo Alto, Calif.).

FIG. 5 shows the differential expression of SEQ ID NO:3 in pancreatic tumor tissue. Tissue specific expression was produced using the LIFESEQ Gold database (Incyte Genomics).

### DESCRIPTION OF THE INVENTION

It must be noted that as used herein and in the appended claims, the singular forms “a”, “an”, and “the” include the plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Definitions

“Biomolecule” refers to a polynucleotide of the present invention, including SEQ ID NOs:1-20 and/or to a protein of the present invention, including SEQ ID NOs:21-23 encoded by SEQ ID NOs:2, 6 and 11.

A “composition” comprises a plurality of polynucleotides, a polynucleotide and a labeling moiety, or a protein and a labeling moiety or pharmaceutical carrier.

“Differential expression” refers to an increased, upregulated or present, or decreased, downregulated or absent, gene expression as detected by presence, absence or at least two-fold changes in the amount of transcribed messenger RNA or translated protein in a sample.

“Diseases associated with matrix-remodeling” include those conditions, diseases and disorders in which the matrix-remodeling occurs, specifically angiogenesis, arthritis, atherosclerosis, cancers, cardiomyopathy, diabetic necrosis, fibrosis, and ulceration.

“Isolated” or “purified” refers to a polynucleotide or protein that is removed from its natural environment and that is separated from other components with which it is naturally present.

“Known matrix-remodeling gene” refers to a gene which has been previously identified as useful in the diagnosis, prognosis, or treatment of diseases associated with matrix-remodeling. The known matrix-remodeling genes are “osteonectin (BM-40), chondroitin/dermatan sulfate proteoglycans (C/DSPG), collagen I, II, II, and IV, connective tissue growth factor (CTGF), fibrillin, fibronectins, fibronectin receptors (fibr-r), fibulin 1, heparan sulfate proteoglycans (HSPG), extracellular matrix protein (hevin), insulin-like growth factor 1 (IGF 1), insulin-like growth factor binding protein (IGFBP), laminin, lumican, matrix Gla protein (MGP), matrix metalloproteases (MMPs), and tissue inhibitors of matrix metalloproteinase 1, 2, and 3 (TIMP 1, 2, and 3)”. Typically, transcripts of the known gene are expressed at higher levels in tissues undergoing matrix-remodeling.

“Labeling moiety” refers to any visible or radioactive label than can be attached to or incorporated into a cDNA or protein. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

A “polynucleotide” whose expression pattern resembles that of a known matrix-remodeling gene can serve as a surrogate marker in the diagnosis, prognosis, or treatment of diseases associated with matrix-remodeling and may be useful in the treatment, or evaluation of treatment, of a disease associated with matrix-remodeling.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue; a tissue print; a fingerprint, buccal cells, skin, or hair; and the like.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “variant” refers to either a polynucleotide or a protein whose sequence diverges from SEQ ID NOs:1-20 or SEQ ID NOs:21-23, respectively. Nucleic acid sequence divergence may result from mutational changes such as deletions, additions, and substitutions of one or more nucleotides; it may also occur because of differences in codon usage. Each of these types of changes may occur alone, or in combination, one or more times in a given sequence. Polypeptide variants include sequences that possess at least one structural or functional characteristic of SEQ ID NOs:21-23.

The Invention

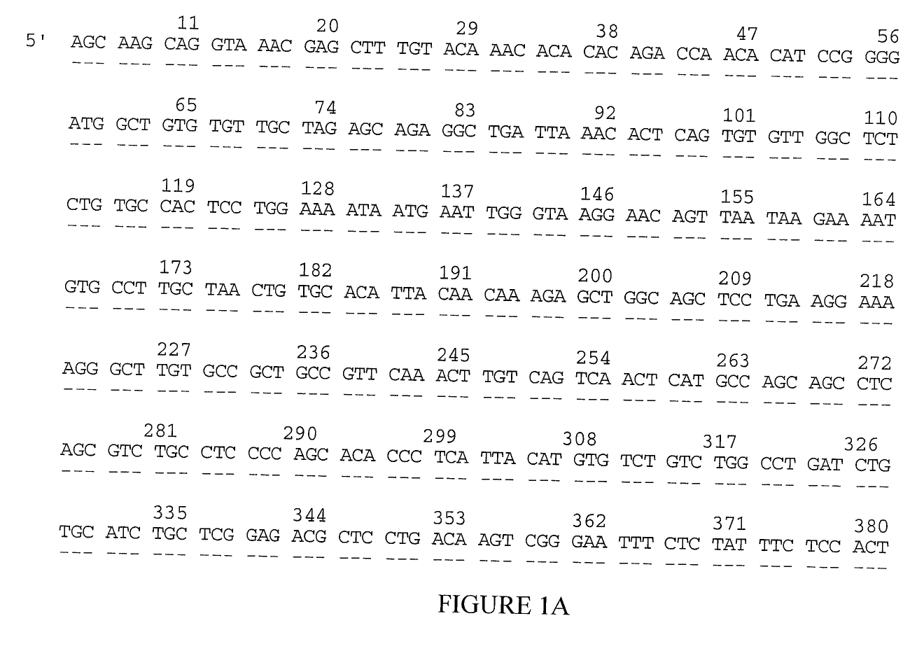

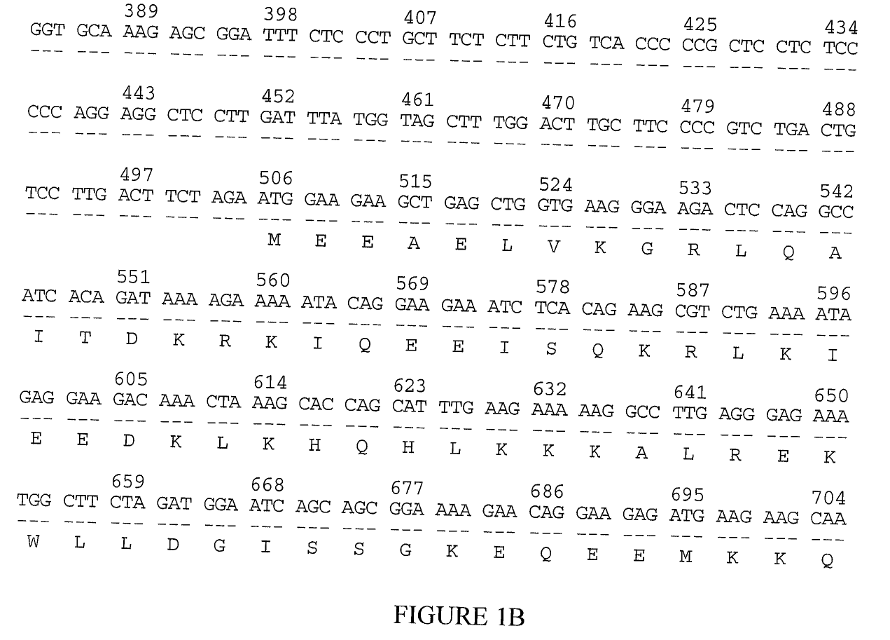

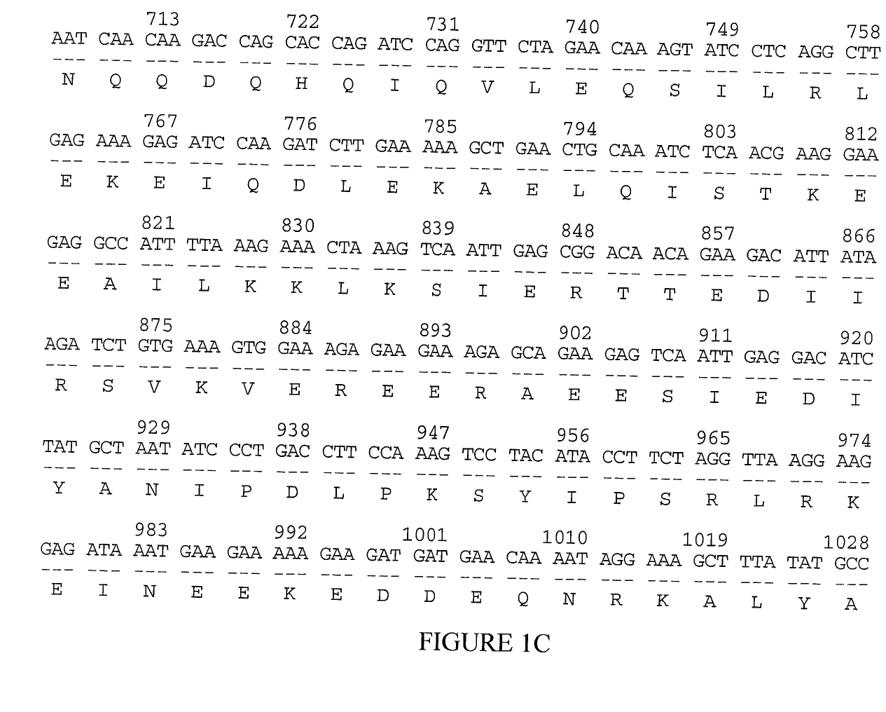

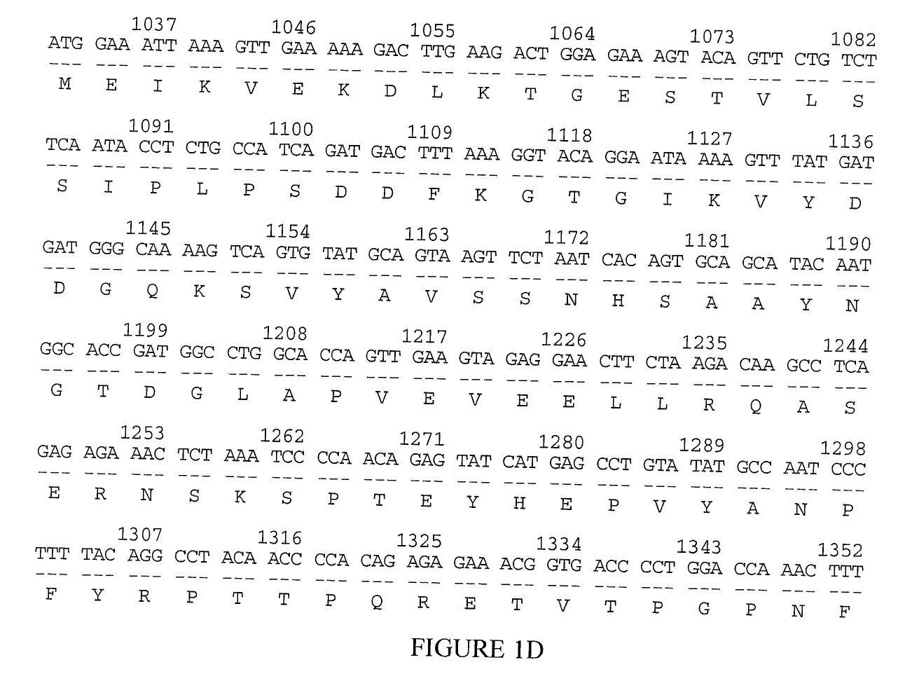

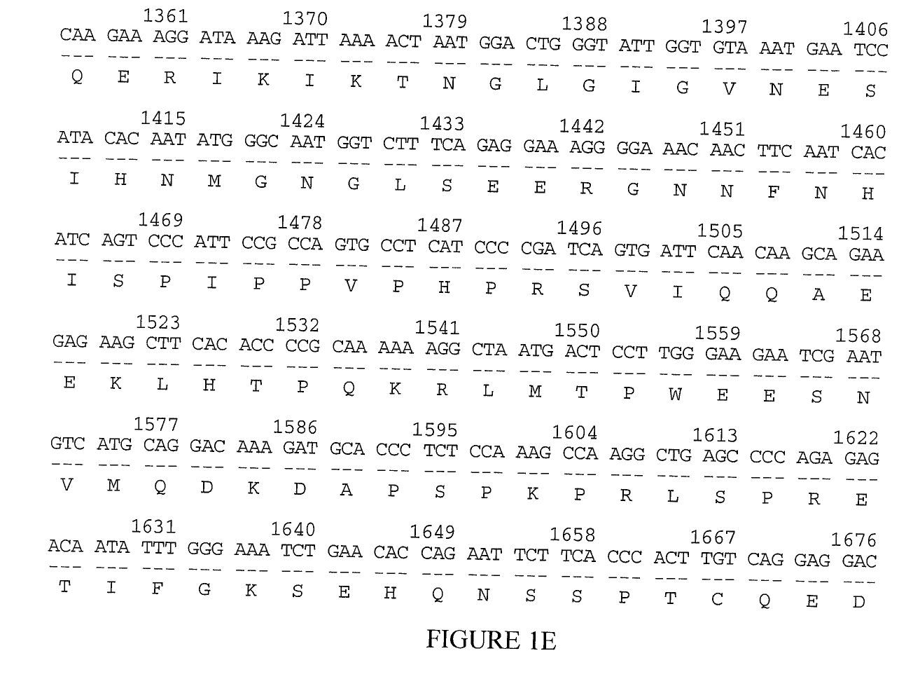

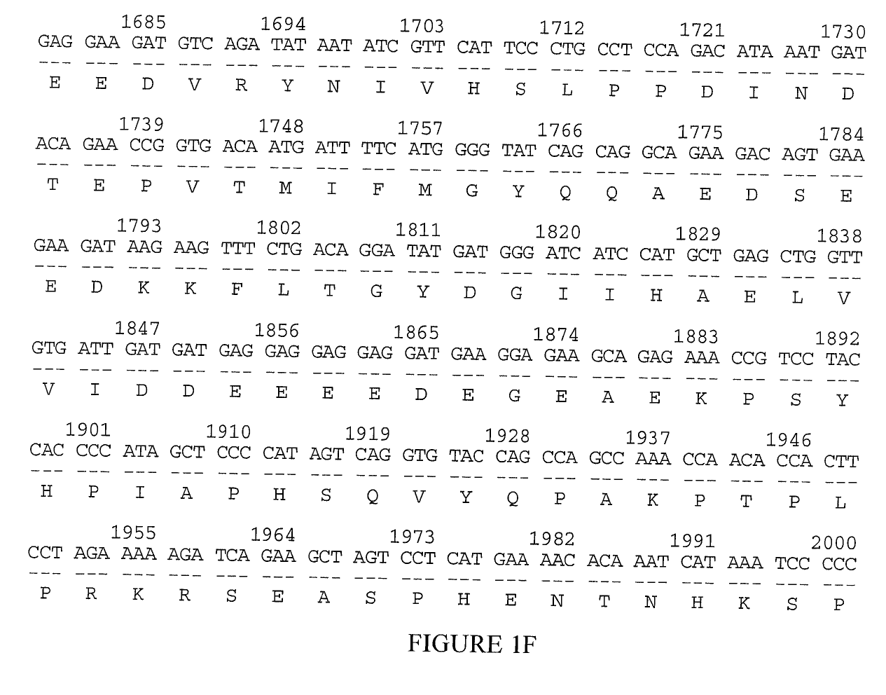

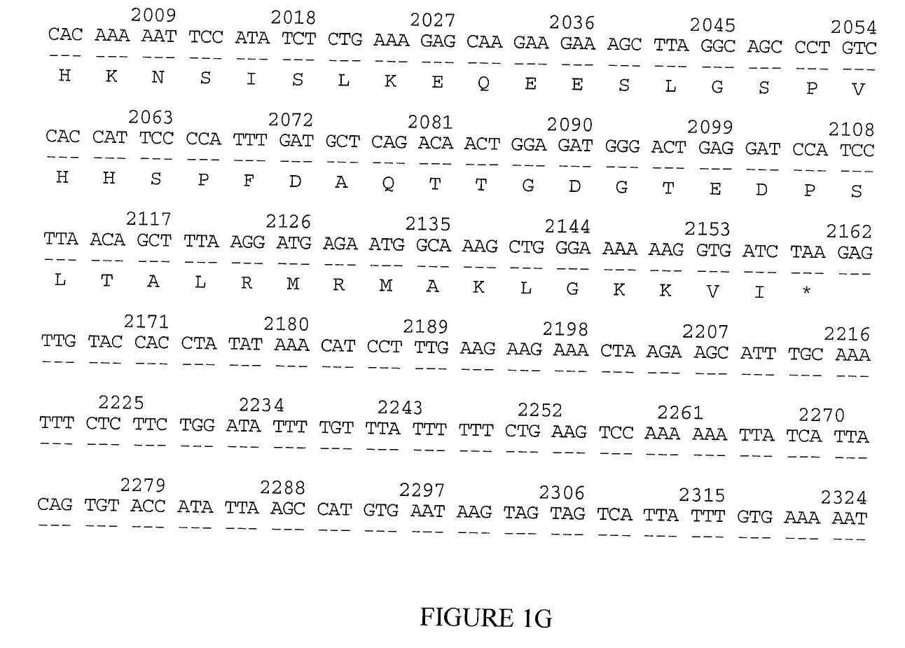

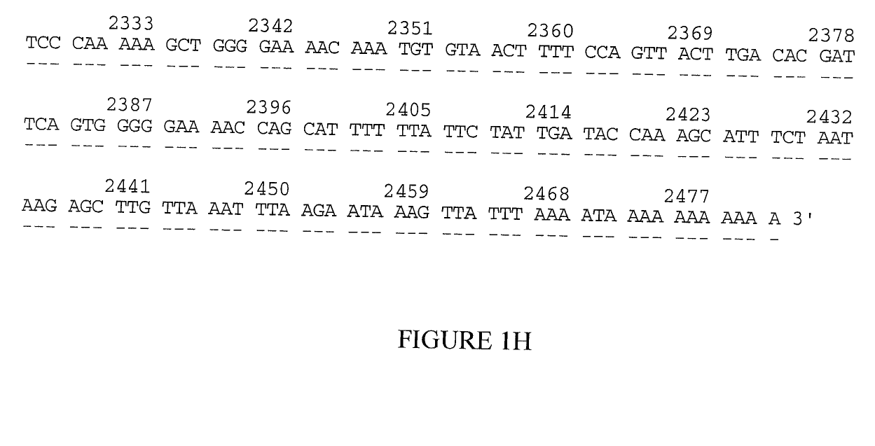

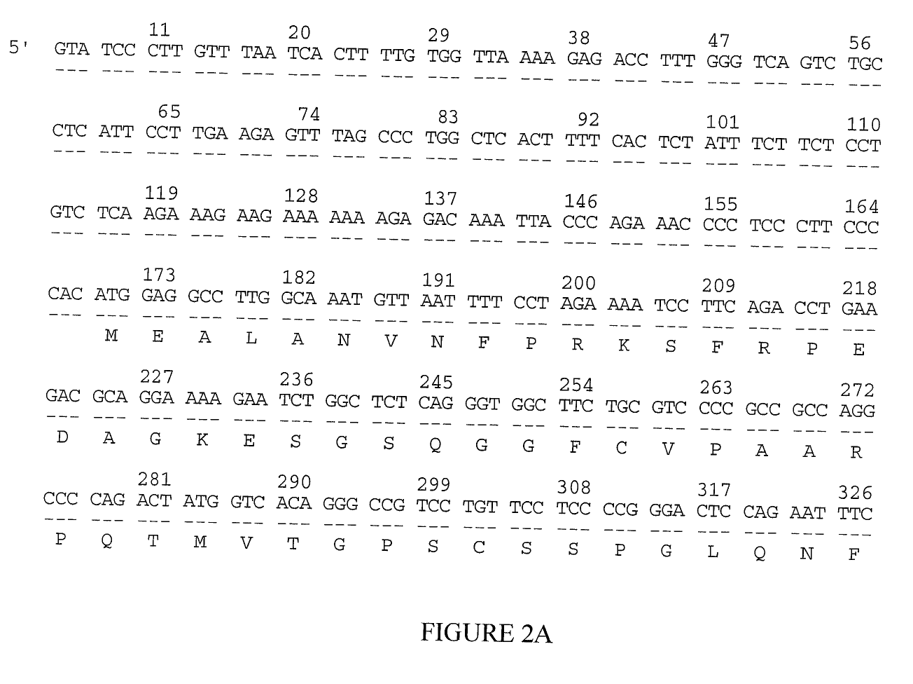

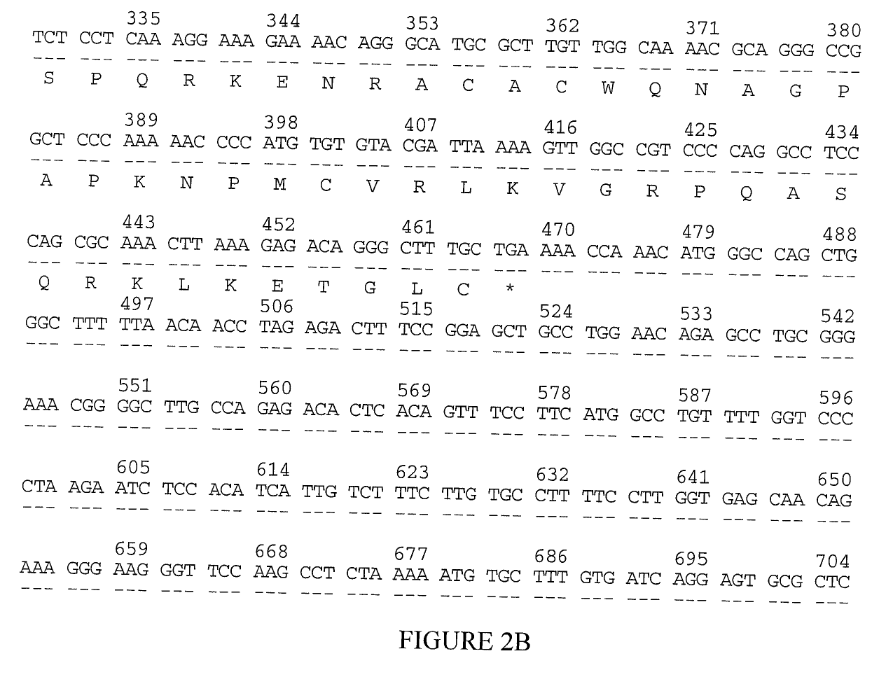

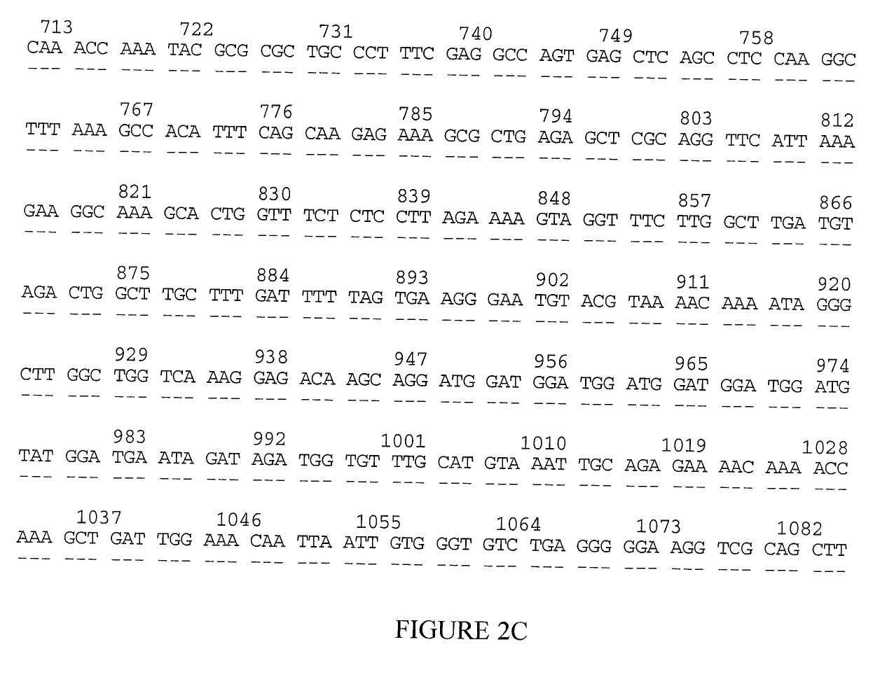

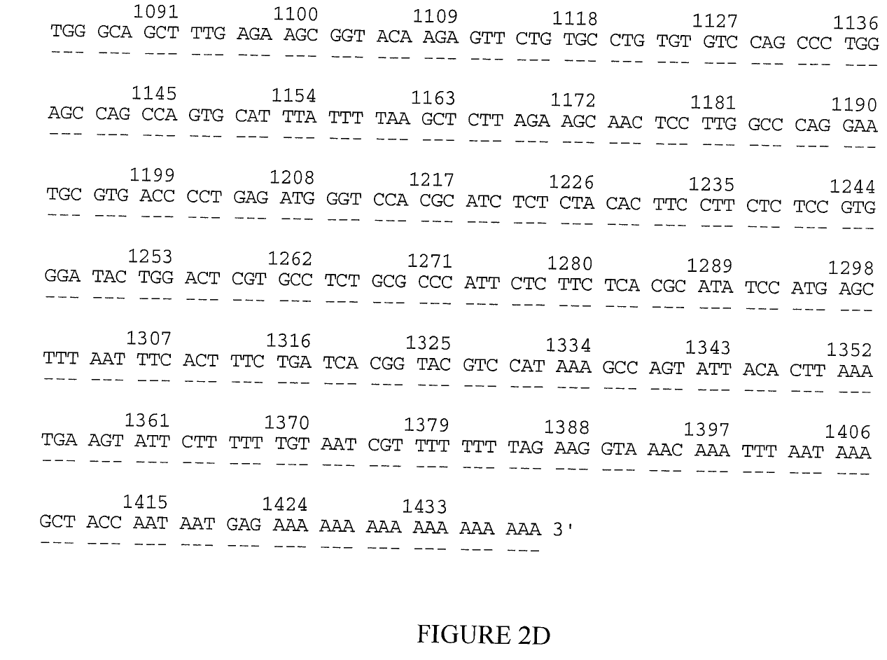

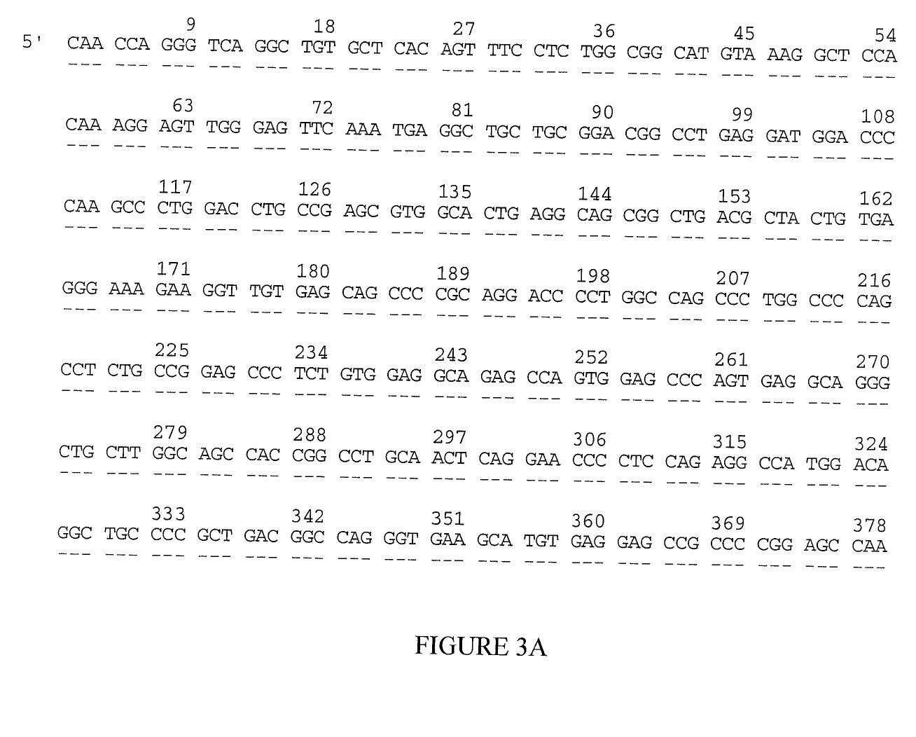

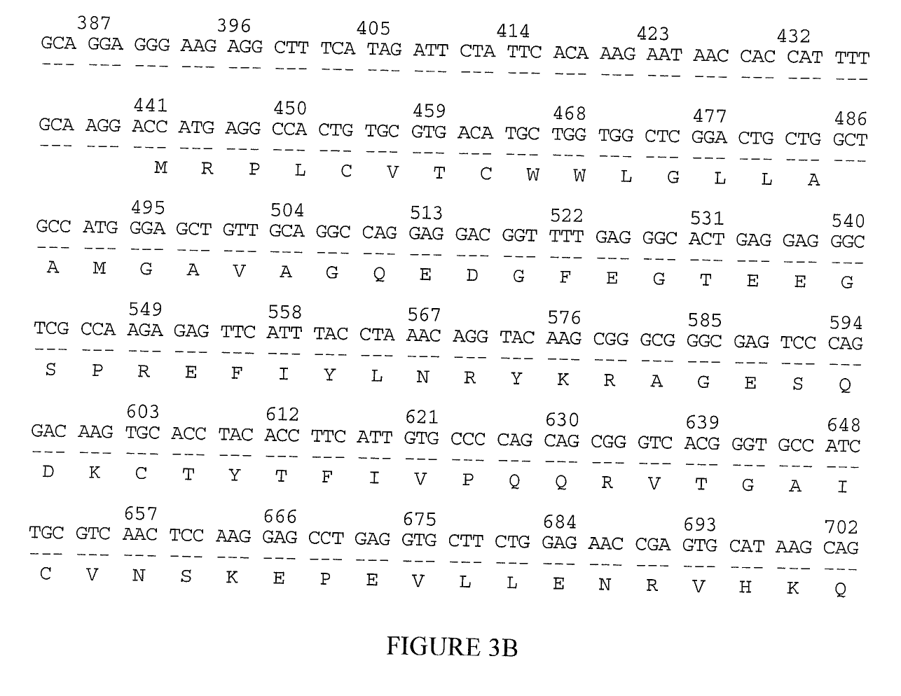

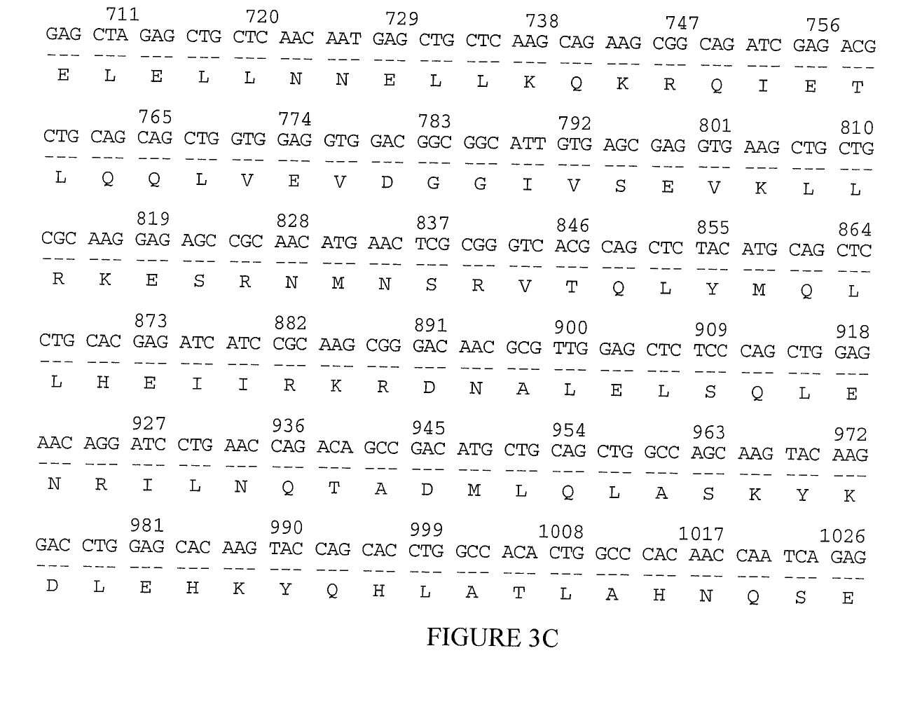

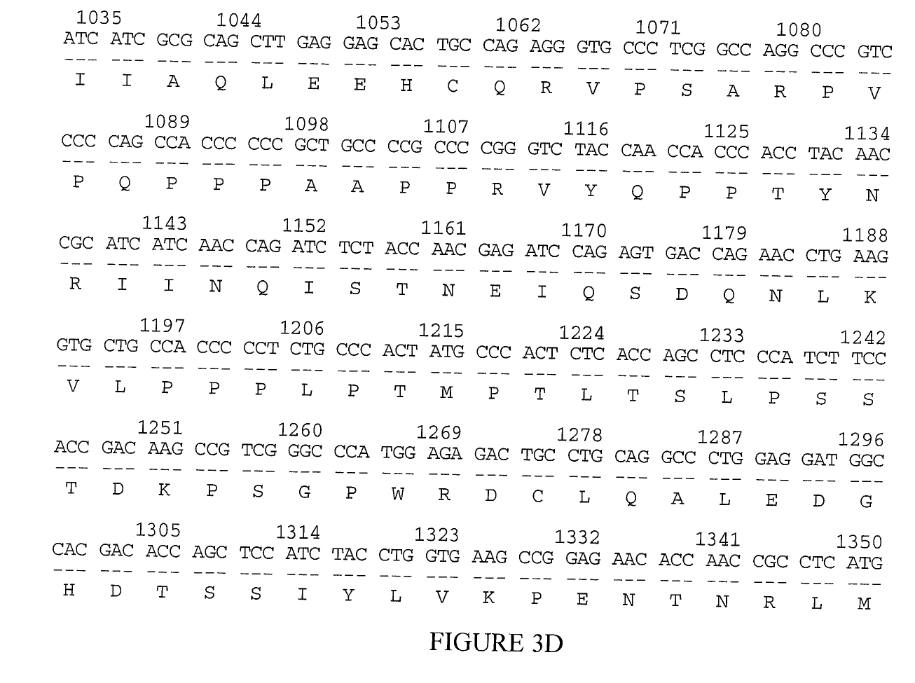

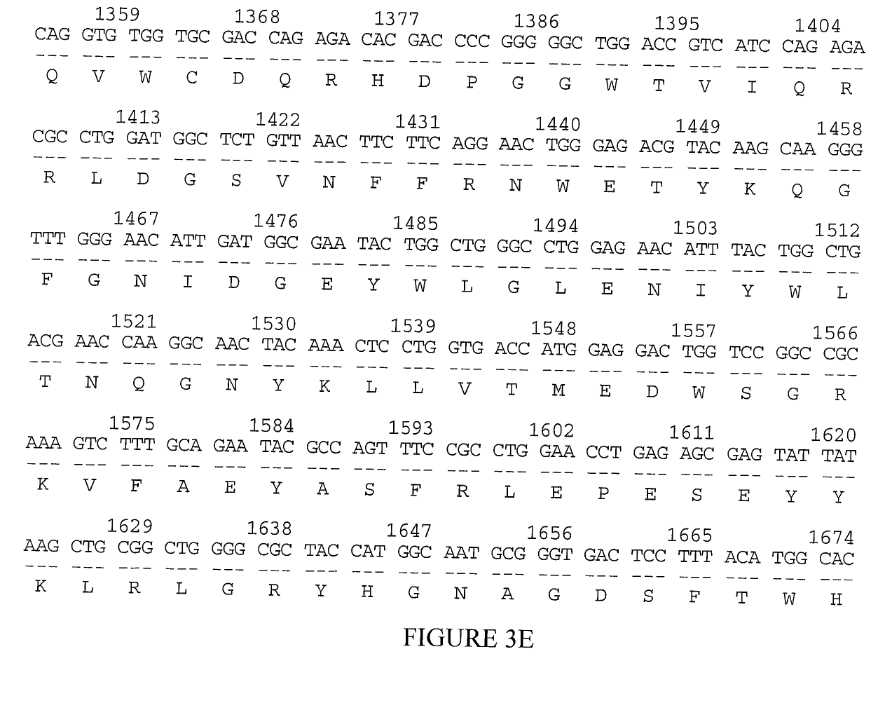

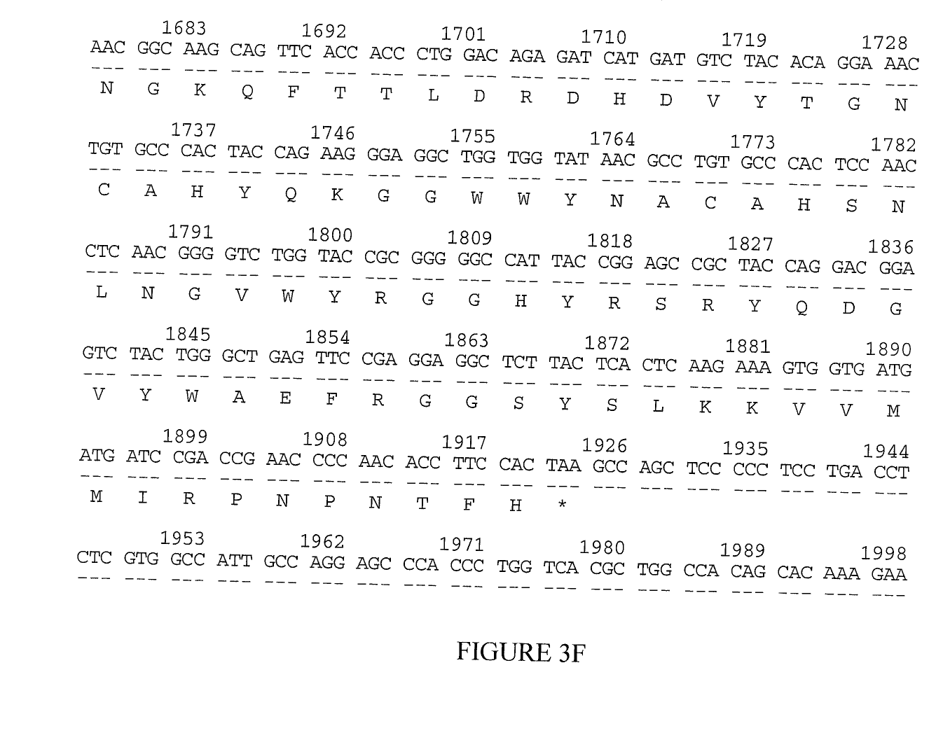

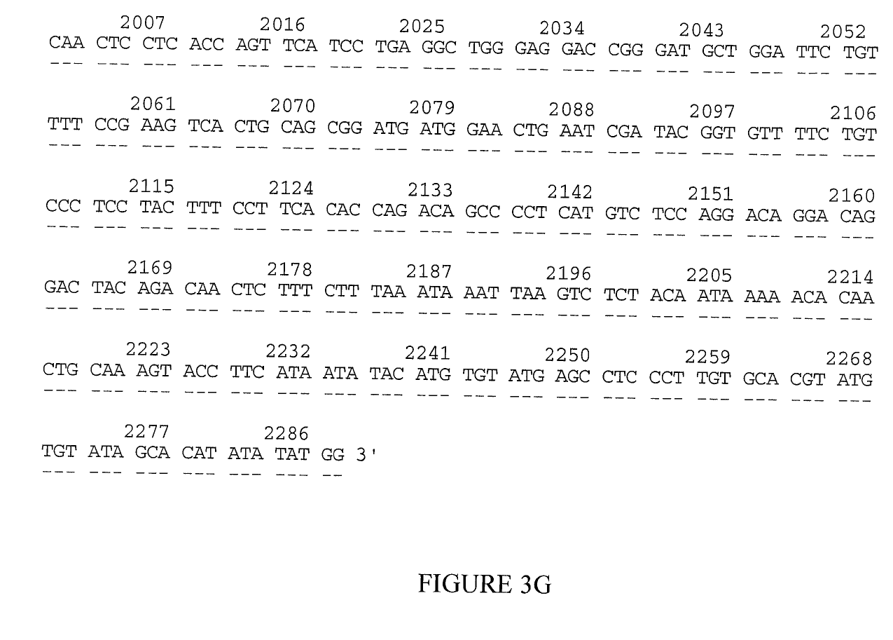

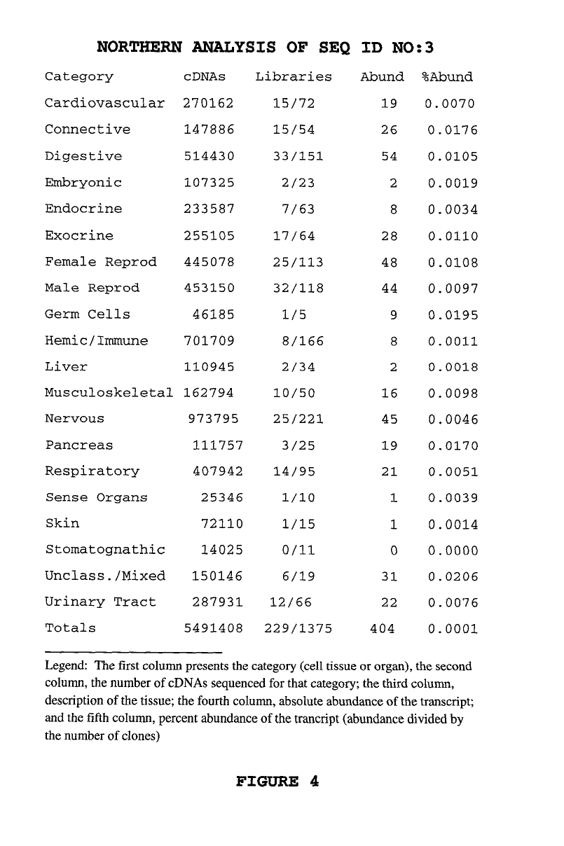

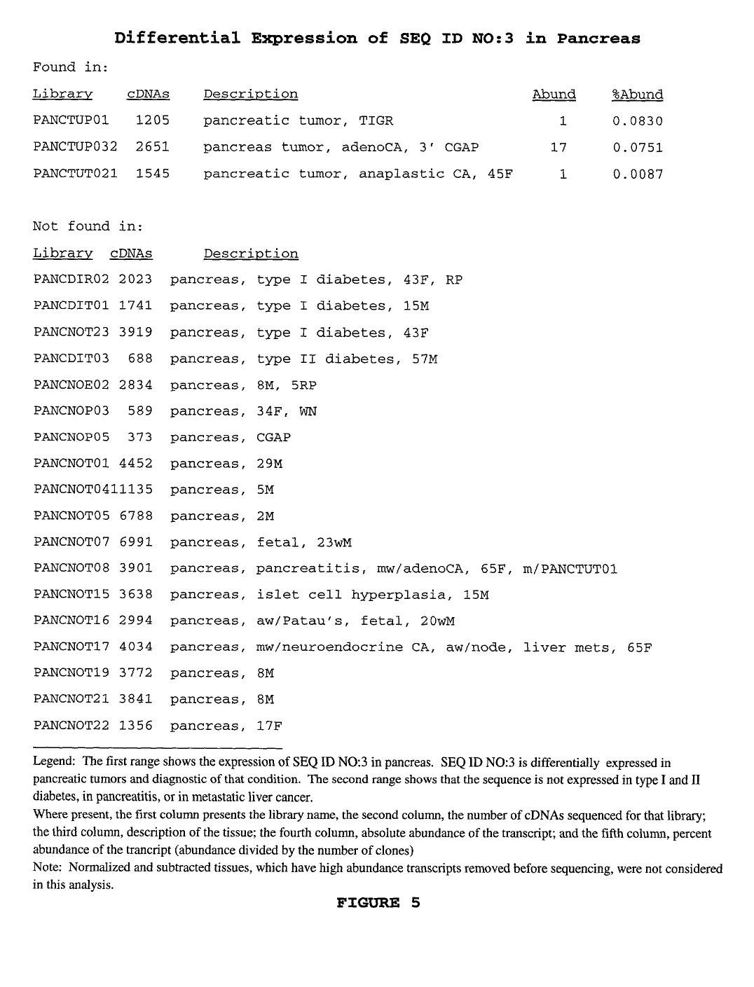

The present invention encompasses a method for identifying biomolecules that are associated with a specific disease, regulatory pathway, subcellular compartment, cell type, tissue type, or species. The method has been named “guilt by association”, and uses known marker genes for a condition, disease or disorder to identify surrogate markers, polynucleotides and proteins that are coexpressed in the same condition, disease, or disorder (Walker and Volkmuth (1999) Prediction of gene function by genome-scale expression analysis: prostate-associated genes. Genome Res 9:1198-1203, incorporated herein by reference). In particular, the method identifies polynucleotides, SEQ ID NOs:1-20 and their encoded polypeptides, SEQ ID NOs: 21-23 (FIGS. 1-3) useful in diagnosis, prognosis, treatment, and evaluation of therapies for diseases associated with matrix-remodeling, particularly, angiogenesis, arthritis, atherosclerosis, cancers, cardiomyopathy, diabetic necrosis, fibrosis, and ulceration. FIGS. 4 and 5 are exemplary of the expression data for each sequence as presented in the LIFESEQ Gold database (Incyte Genomics).

The method provides first identifying polynucleotides that are expressed in a plurality of cDNA libraries. The identified polynucleotides include unknown polynucleotides and polynucleotides of known function which are specifically expressed in a particular disease process, subcellular compartment, cell type, tissue type, or species. The expression patterns of the known matrix-remodeling genes are compared with those of the polynucleotides of unknown function to determine whether a specified coexpression probability threshold is met. Through this comparison, a subset of the polynucleotides of unknown function having a high coexpression probability with the known marker genes can be identified. The high coexpression probability correlates with a particular coexpression probability threshold which is less than 0.001, and more preferably less than 0.00001.

The polynucleotides originate from cDNA libraries derived from a variety of sources including, but not limited to, eukaryotes such as human, mouse, rat, dog, monkey, plant, and yeast and prokaryotes such as bacteria and viruses. These polynucleotides can also be selected from a variety of sequence types including, but not limited to, expressed sequence tags (ESTs), assembled polynucleotide sequences, exons, introns, 5′ untranslated regions, and 3′ untranslated regions. To have statistically significant analytical results, the polynucleotides need to be expressed in at least three cDNA libraries.

The cDNA libraries used in the coexpression analysis of the present invention can be obtained from blood vessels, heart, blood cells, cultured cells, connective tissue, epithelium, islets of Langerhans, neurons, phagocytes, biliary tract, esophagus, stomach, duodenum, ileum, colon, liver, pancreas, fetus, placenta, chromaffin system, endocrine glands, ovary, uterus, penis, prostate, seminal vesicles, testis, bone marrow, lymph nodes, cartilage, muscles, skeleton, brain, ganglia, neuroglia, neurosecretory system, peripheral nervous system, bronchus, larynx, lung, nose, pleurus, ear, eye, mouth, pharynx, exocrine glands, bladder, kidney, ureter, and the like. The number of cDNA libraries selected can range from as few as 20 to greater than 10,000. Preferably, the number of the cDNA libraries is greater than 500.

In a preferred embodiment, the polynucleotides are assembled sequence fragments derived from a single transcript. Assembly of the sequences can be performed using sequences of various types including, but not limited to, ESTs, extensions, or shotgun sequences. In a most preferred embodiment, the polynucleotides are derived from human sequences that have been assembled using the algorithm disclosed in “Database and System for Storing, Comparing and Displaying Related Biomolecular Sequence Information”, U.S. Ser. No. 9,276,534, filed Mar. 25, 1999, incorporated herein by reference.

Experimentally, differential expression of the polynucleotides can be evaluated by methods including, but not limited to, differential display by spatial immobilization or by gel electrophoresis, genome mismatch scanning, representational difference analysis, and transcript imaging. Additionally, differential expression can be assessed by microarray technology. These methods may be used alone or in combination.

Known matrix-remodeling genes can be selected from research and medical literature based on their use as diagnostic or prognostic markers or as therapeutic targets for diseases associated with matrix-remodeling. Preferably, the known matrix-remodeling genes include BM-40, C/DSPG, collagen I, II, II, and IV, CTGF, fibrillin, fibronectins, fibr-r, fibulin 1, HSPG, hevin, IGF 1, IGFBP, laminin, lumican, MGP, MMPs, TIMP 1, 2, and 3, and the like.

The procedure for identifying novel polynucleotides that exhibit a statistically significant coexpression pattern with known matrix-remodeling genes is as follows. First, the presence or absence of a gene or polynucleotide in a cDNA library is defined: a gene of polynucleotide is present in a cDNA library when at least one fragment corresponding to that gene or polynucleotide is detected in a sample taken from the library, and a gene or polynucleotide is absent from a library when no corresponding cDNA fragment is detected in the sample.

Second, the significance of coexpression is evaluated using a probability method to measure a due-to-chance probability of the coexpression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York N.Y.; Rice (1988) Mathematical Statistics and Data Analysis, Duxbury Press, Pacific Grove Calif.). A Bonferroni correction (Rice, supra, page 384) can also be applied in combination with one of the probability methods for correcting statistical results of one gene or polynucleotide versus multiple other genes or polynucleotides. In a preferred embodiment, the due-to-chance probability is measured by a Fisher exact test, and the threshold of the due-to-chance probability is set to less than 0.001, and the probability is more preferably less than 0.00001.

To determine whether two genes, A and B, have similar coexpression patterns, occurrence data vectors can be generated as illustrated in Table 1, wherein a gene's presence is indicated by a one and its absence by a zero. A zero indicates that the gene did not occur in the library, and a one indicates that it occurred at least once.

TABLE 1
Occurrence data for genes A and B

| | Library 1 | Library 2 | Library 3 . . . Library N |
|---|---|---|---|
| gene A | 1 | 1 | 0 . . . 0 |
| gene B | 1 | 0 | 1 . . . 0 |

For a given pair of genes, the occurrence data in Table 1 can be summarized in a 2×2 contingency table.

TABLE 2
Contingency table for co-occurrences of genes A and B

| | Gene A present | Gene A absent | Total |
|---|---|---|---|
| Gene B present | 8 | 2 | 10 |
| Gene B absent | 2 | 18 | 20 |
| Total | 10 | 20 | 30 |

Table 2 presents co-occurrence data for gene A and gene B in a total of 30 libraries. Both gene A and gene B occur 10 times in the libraries. Table 2 summarizes and presents 1) the number of times gene A and B are both present in a library, 2) the number of times gene A and B are both absent in a library, 3) the number of times gene A is present while gene B is absent, and 4) the number of times gene B is present while gene A is absent. The upper left entry is the number of times the two genes co-occur in a library, and the middle right entry is the number of times neither gene occurs in a library. The off diagonal entries are the number of times one gene occurs while the other does not. Both A and B are present eight times and absent 18 times, gene A is present while gene B is absent two times, and gene B is present while gene A is absent two times. The probability (“p-value”) that the above association occurs due to chance as calculated using a Fisher exact test is 0.0003. Associations are generally considered significant if a p-value is less than 0.01 (Agresti, supra; Rice, supra).

This method of estimating the probability for coexpression of two genes makes several assumptions. The method assumes that the libraries are independent and are identically sampled. However, in practical situations, the selected cDNA libraries are not entirely independent because more than one library may be obtained from a single patient or tissue, and they are not entirely identically sampled because different numbers of cDNAs may be sequenced from each library (typically ranging from 5,000 to 10,000 cDNAs per library). In addition, because a Fisher exact coexpression probability is calculated for each gene or polynucleotide versus 41,419 other genes or polynucleotides, a Bonferroni correction for multiple statistical tests is necessary.

Using the method of the present invention, we have identified 20 novel polynucleotides that exhibit strong association, or coexpression, with known genes that are matrix-remodeling-specific. These known matrix-remodeling genes include BM-40, C/DSPG, collagen I, II, II, and IV, CTGF, fibrillin, fibronectins, fibr-r, fibulin 1, HSPG, hevin, IGF 1, IGFBP, laminin, lumican, MGP, MMPs, TIMP 1, 2, and 3. The results presented in Tables 3 and 4 show that the expression of the 20 novel polynucleotides have direct or indirect association with the expression of known matrix-remodeling genes. Therefore, the novel polynucleotides can potentially be used in diagnosis, prognosis, or treatment of diseases associated with matrix-remodeling, or in the evaluation of therapies for diseases associated with matrix-remodeling. Further, the proteins encoded by the 20 novel polynucleotides are potential therapeutic proteins or targets for identifying therapeutics against diseases associated with matrix-remodeling.

Therefore, in one embodiment, the present invention encompasses a polynucleotide comprising a nucleic acid sequence selected from SEQ ID NOs:1-20. These 20 polynucleotides are shown by the method of the present invention to have strong coexpression association with known matrix-remodeling genes and with each other. The invention also encompasses a variant of the polynucleotide or its complement.

One preferred method for identifying variants entails using the polynucleotide or the encoded protein to search against the GenBank primate (pri), rodent (rod), and mammalian (mam), vertebrate (vrtp), and eukaryote (eukp) databases, SwissProt, BLOCKS (Bairoch et al. (1997) Nucleic Acids Res 25:217-221), PFAM, and other databases that contain previously identified and annotated motifs, sequences, and gene functions. Methods that search for primary sequence patterns with secondary structure gap penalties (Smith et al. (1992) Protein Engineering 5:35-51) as well as algorithms such as BLAST (Basic Local Alignment Search Tool; Altschul (1993) J Mol Evol 36:290-300; and Altschul et al. (1990) J Mol Biol 215:403-410), BLOCKS (Henikoff and Henikoff (1991) Nucleic Acids Res 19:6565-6572), Hidden Markov Models (HMM; Eddy (1996) Cur Opin Str Biol 6:361-365; Sonnhammer et al. (1997) Proteins 28:405-420), and the like, can be used to manipulate and analyze nucleotide and amino acid sequences. These databases, algorithms and other methods are well known in the art and are described in Ausubel et al. (1997; Short Protocols in Molecular Biology, John Wiley & Sons, New York N.Y.) and in Meyers (1995; Molecular Biology and Biotechnology, Wiley VCH, New York N.Y., pp. 856-853).

Also encompassed by the invention are polynucleotides that are capable of hybridizing to SEQ ID NOs:1-20, and fragments thereof, under stringent conditions. Stringent conditions can be defined by salt concentration, temperature, and other chemicals and conditions well known in the art. In particular, stringency can be increased by reducing the concentration of salt, or raising the hybridization temperature. Varying additional parameters, such as hybridization time, the concentration of detergent or solvent, and the inclusion or exclusion of carrier DNA, are well known to those skilled in the art. Additional variations on these conditions will be readily apparent to those skilled in the art (Wahl and Berger (1987) Methods Enzymol 152:399-407; Kimmel (1987) Methods Enzymol 152:507-511; Ausubel (supra); and Sambrook et al. (1989) Molecular Cloning, A Laboratory Manual, Cold Spring Harbor Press, Plainview N.Y.).

The polynucleotide can be extended utilizing a partial nucleic acid sequence and employing various PCR-based methods known in the art to detect upstream sequences, such as promoters and regulatory elements (Dieffenbach and Dveksler (1995) PCR Primer, a Laboratory Manual, Cold Spring Harbor Press, Plainview N.Y.; Sarkar (1993) PCR Methods Applic 2:318-322; Triglia et al. (1988) Nucleic Acids Res 16:8186; Lagerstrom et al. (1991) PCR Methods Applic 1:111-119; and Parker et al. (1991) Nucleic Acids Res 19:3055-306). Additionally, one may use PCR, nested primers, and PROMOTERFINDER libraries (Clontech, Palo Alto, Calif.) to walk genomic DNA. This procedure avoids the need to screen libraries and is useful in finding intron/exon junctions. For all PCR-based methods, primers may be designed using commercially available software, such as OLIGO primer analysis software (Molecular Biology Insights, Cascade Colo.) or another appropriate program, to be about 18 to 30 nucleotides in length, to have a GC content of about 50% or more, and to anneal to the template at temperatures of about 68° C. to 72° C.

In another aspect of the invention, the polynucleotide encoding the protein can be cloned in recombinant DNA molecules that direct expression of the protein in appropriate host cells. Due to the inherent degeneracy of the genetic code, other DNA sequences which encode the same or a functionally equivalent amino acid sequence may be produced and used to express the protein encoded by the polynucleotide. The nucleotide sequences of the present invention can be engineered using methods generally known in the art in order to alter the nucleotide sequences for a variety of purposes including, but not limited to, modification of the cloning, processing, and/or expression of the protein. DNA shuffling by random fragmentation and PCR reassembly of polynucleotide fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

In order to express a biologically active protein encoded by the polynucleotide, the coding sequence may be inserted into an appropriate expression vector containing elements for transcriptional and translational control of the inserted sequence in a host. These elements include, preferably host specific, regulatory sequences, such as enhancers, constitutive and inducible promoters, and 5′ and 3′ untranslated regions engineered or introduced into the vector. Methods which are well known to those skilled in the art may be used to construct expression vectors containing the polynucleotide encoding a matrix-remodeling protein and appropriate transcriptional and translational control elements. These methods include in vitro recombinant DNA techniques, synthetic techniques, and in vivo genetic recombination (Sambrook, supra and Ausubel, supra).

A variety of expression vector/host cell systems may be utilized to contain and express the polynucleotide. These include, but are not limited to, microorganisms such as bacteria transformed with recombinant bacteriophage, plasmid, or cosmid DNA expression vectors; yeast transformed with yeast expression vectors; insect cell systems infected with viral expression vectors (baculovirus); plant cell systems transformed with viral expression vectors, cauliflower mosaic virus (CaMV) or tobacco mosaic virus (TMV), or with bacterial expression vectors (Ti or pBR322 plasmids); or animal cell systems. The invention is not limited by the host cell employed. For long term production of recombinant proteins in mammalian systems, stable expression of a protein in cell lines is preferred. For example, polynucleotides encoding SEQ ID NO:21-23 can be transformed into cell lines using expression vectors which may contain viral origins of replication and/or endogenous expression elements and a selectable marker gene on the same or on a separate vector.

In general, host cells that contain the polynucleotide and that express the protein may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences. Immunological methods for detecting and measuring the expression of a protein using either specific polyclonal or monoclonal antibodies are known in the art. Examples of such techniques include enzyme-linked immunosorbent assays (ELISAs), radioimmunoassays (RIAs), and fluorescence activated cell sorting (FACS).

Host cells transformed with a polynucleotide of the invention may be cultured under conditions for the expression and recovery of the protein from cell culture. The protein produced by a transformed cell may be secreted or retained intracellularly depending on the sequence and/or the vector used. As will be understood by those of skill in the art, expression vectors containing polynucleotides of the invention may be designed to contain signal sequences which direct secretion of the protein encoded by the polynucleotide through a prokaryotic or eukaryotic cell membrane.

In addition, a host cell strain may be chosen for its ability to modulate expression of the inserted sequences or to process the expressed protein in the desired fashion. Such modifications of the protein include, but are not limited to, acetylation, carboxylation, glycosylation, phosphorylation, lipidation, and acylation. Post-translational processing which cleaves a “prepro” form of the protein may also be used to specify protein targeting, folding, and/or activity. Different host cells which have specific cellular machinery and characteristic mechanisms for post-translational activities (e.g., CHO, HeLa, MDCK, HEK293, and WI38), are available from the American Type Culture Collection (ATCC, Manassas Va.) and may be chosen to ensure the correct modification and processing of the foreign protein.

In another embodiment of the invention, natural, modified, or recombinant polynucleotide of the invention is ligated to a heterologous sequence resulting in translation of a fusion protein containing heterologous protein moieties in any of the aforementioned host systems. Such heterologous protein moieties facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, hemagglutinin (HA) and monoclonal antibody epitopes.

In another embodiment, the polynucleotides are synthesized, in whole or in part, using chemical methods well known in the art (Caruthers et al. (1980) Nucleic Acids Symp Ser (7) 215-223; Horn et al. (1980) Nucleic Acids Symp Ser (7) 225-232; and Ausubel, supra). Alternatively, the encoded protein may be synthesized using chemical methods. For example, peptide synthesis can be performed using various solid-phase techniques (Roberge et al. (1995) Science 269:202-204). Automated synthesis may be achieved using the 431A peptide synthesizer (Applied Biosystems (ABI), Foster City Calif.). Additionally, the protein, or any portion thereof, may be altered during direct synthesis and/or combined with sequences from other proteins, or any part thereof, to produce a variant.

In another embodiment, the invention provides a purified protein comprising the amino acid sequence selected from the group consisting of SEQ ID NOs:21-23 or fragments thereof.

Screening, Diagnostics and Therapeutics

The sequences of the these polynucleotides can be used as surrogate markers in diagnosis, prognosis, treatment, and evaluation of therapies for diseases in which matrix-remodeling occurs. Further, the proteins and peptides encoded by the polynucleotides can be used in diagnostic assays including PAGE and Western analyses, and they are potential therapeutic proteins and/or targets for discovering drugs that can be used to treat diseases associated with matrix-remodeling.

The polynucleotides may be used to screen a plurality of molecules and compounds for specific binding affinity. The assay can be used to screen a plurality of DNA molecules, RNA molecules, peptide nucleic acids, peptides, ribozymes, antibodies, agonists, antagonists, immunoglobulins, inhibitors, proteins including transcription factors, enhancers, repressors, and drugs and the like which regulate the activity of the polynucleotide in the biological system. The assay involves providing a plurality of molecules and compounds, combining the polynucleotide or a composition of the invention with the plurality of molecules and compounds under conditions suitable to allow specific binding, and detecting specific binding to identify at least one molecule or compound which specifically binds the polynucleotide.

Similarly the proteins or portions thereof may be used to screen libraries of molecules or compounds in any of a variety of screening assays. The portion of a protein employed in such screening may be free in solution, affixed to an abiotic or biotic substrate (e.g. borne on a cell surface), or located intracellularly. Specific binding between the protein and the molecule may be measured. The assay can be used to screen a plurality of DNA molecules, RNA molecules, PNAs, peptides, mimetics, ribozymes, antibodies, agonists, antagonists, immunoglobulins, inhibitors, peptides, polypeptides, drugs and the like, which specifically bind the protein. One method for high throughput screening using very small assay volumes and very small amounts of test compound is described in Burbaum et al. U.S. Pat. No. 5,876,946, incorporated herein by reference, which screens large numbers of molecules for enzyme inhibition or receptor binding.

In one preferred embodiment, the polynucleotide is used for diagnostic purposes as a probe to determine the absence, presence, or altered—increased or decreased compared to a normal standard—expression of the gene. The polynucleotides comprise complementary RNA and DNA molecules, branched nucleic acids, and/or peptide nucleic acids (PNAs). Alternatively, the polynucleotides are used to detect and quantitate gene expression in samples in which expression of the polynucleotide is correlated with disease. In another alternative, the polynucleotides can be used to detect genetic polymorphisms associated with a disease. These polymorphisms may be detected in a transcript, cDNA or genomic sequence.

The specificity of the probe is determined by whether it is made from a unique region, a regulatory region, or from a conserved motif. Both probe specificity and the stringency of diagnostic hybridization or amplification (maximal, high, intermediate, or low) will determine whether the probe identifies only naturally occurring, exactly complementary sequences, allelic variants, or related sequences. Probes designed to detect related sequences should preferably have at least 50% sequence identity to any of the polynucleotides encoding the protein.

Methods for producing hybridization probes include the cloning of nucleic acid sequences into vectors for the production of RNA probes. Such vectors are known in the art, are commercially available, and may be used to synthesize RNA probes in vitro by adding RNA polymerases and labeled nucleotides. Hybridization probes may labeled using either visible or radioactive moieties. These moieties are well known in the art. The labeled polynucleotides may be used in Southern or northern analysis, dot/slot blot, or other membrane-based technologies; in PCR technologies; and in microarrays utilizing fluids or tissues to detect altered transcript expression.

Polynucleotides can be labeled by standard methods and added to a sample from a subject under conditions for the formation of hybridization complexes. After incubation, the sample is washed, and the signal associated with hybrid complex formation is quantitated and compared with a standard value. Standard values are derived from any control sample, typically one that is free of the suspect disease. If the amount of signal in a subject sample is altered in comparison to the standard value, then the presence of altered levels of expression indicates the presence of the disease. Qualitative and quantitative methods for comparing the hybridization complexes formed in subject samples with previously established standards are well known in the art.

Once the presence of a disease is established and a treatment protocol is initiated, hybridization or amplification assays can be repeated on a regular basis to determine if the level of expression in the patient begins to approximate that which is observed in a healthy subject. The results obtained from successive assays may be used to show the efficacy of treatment over a period ranging from several days to many years.

The polynucleotides may be used for the diagnosis of a variety of diseases associated with matrix-remodeling including cancers such as adenocarcinoma, leukemia, lymphoma, melanoma, myeloma, sarcoma, teratocarcinoma, and, in particular, cancers or tumors of the adrenal gland, bladder, bone, bone marrow, brain, breast, cervix, gall bladder, ganglia, gastrointestinal tract, heart, kidney, liver, lung, muscle, nerve, ovary, pancreas, parathyroid, penis, prostate, salivary glands, skin, spleen, testis, thymus, thyroid, and uterus.

The polynucleotides may also be used on a substrate such as microarray to monitor the expression patterns. The microarray may also be used to identify splice variants, mutations, and polymorphisms. Information derived from analyses of the expression patterns may be used to determine gene function, to understand the genetic basis of a disease, to diagnose a disease, and to develop and monitor the activities of therapeutic agents used to treat a disease. Microarrays may also be used to detect genetic diversity, single nucleotide polymorphisms which may characterize a particular population, at the genome level.

In yet another alternative, polynucleotides may be used to generate hybridization probes useful in mapping the naturally occurring genomic sequence. Fluorescent in situ hybridization (FISH) may be correlated with other physical chromosome mapping techniques and genetic map data as described in Heinz-Ulrich et al. (In: Meyers, supra, pp. 965-968).

In another embodiment, antibodies or Fabs comprising an antigen binding site that specifically bind the protein may be used for the diagnosis of diseases characterized by the over-or-underexpression of the protein. A variety of protocols for measuring protein expression including ELISAs, RIAs, and FACS, are well known in the art and provide a basis for diagnosing altered or abnormal levels of the protein expression. Standard values for protein expression are established by combining samples taken from healthy subjects, preferably human, with antibody which specifically binds to the protein under conditions for complex formation. The amount of complex formation may be quantitated by various methods, preferably by photometric means. Quantities of protein expressed in disease samples, from biopsied tissues, are compared with standard values. Deviation between standard and subject values establishes the parameters for diagnosing or monitoring disease. Alternatively, one may use competitive drug screening assays in which neutralizing antibodies capable of specifically binding the protein compete with a test compound for binding sites. Antibodies can also be used to detect the presence of any peptide which shares one or more antigenic determinants with the protein. In one aspect, the antibodies of the present invention can be used for treatment or for monitoring therapeutic treatment of diseases associated with matrix-remodeling.

In another aspect, the cDNA, or its complement, may be used therapeutically for the purpose of expressing mRNA and protein, or conversely to block transcription or translation of the mRNA. Expression vectors may be constructed using elements from retroviruses, adenoviruses, herpes or vaccinia viruses, or bacterial plasmids, and the like. These vectors may be used for delivery of nucleotide sequences to a particular target organ, tissue, or cell population. Methods well known to those skilled in the art can be used to construct vectors to express nucleic acid sequences or their complements. (See, e.g., Maulik et al. (1997) Molecular Biotechnology, Therapeutic Applications and Strategies, Wiley-Liss, New York N.Y.) Alternatively, the cDNA or its complement, may be used for somatic cell or stem cell gene therapy. Vectors may be introduced in vivo, in vitro, and ex vivo. For ex vivo therapy, vectors are introduced into stem cells taken from the subject, and the resulting transgenic cells are clonally propagated for autologous transplant back into that same subject. Delivery of the cDNA by transfection, liposome injections, or polycationic amino polymers may be achieved using methods which are well known in the art (Goldman et al. (1997) Nature Biotechnology 15:462-466). Additionally, endogenous gene expression may be inactivated using homologous recombination methods which insert an inactive gene sequence into the coding region or other targeted region of the cDNA (Thomas et al. (1987) Cell 51: 503-512).

Vectors containing the cDNA can be transformed into a cell or tissue to express a missing protein or to replace a nonfunctional protein. Similarly a vector constructed to express the complement of the cDNA can be transformed into a cell to downregulate the protein expression. Complementary or antisense sequences may consist of an oligonucleotide derived from the transcription initiation site; nucleotides between about positions −10 and +10 from the ATG are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee et al. In: Huber and Carr (1994) Molecular and Immunologic Approaches, Futura Publishing, Mt. Kisco N.Y., pp. 163-177).

Ribozymes, enzymatic RNA molecules, may also be used to catalyze the cleavage of mRNA and decrease the levels of particular mRNAs, such as those comprising the cDNAs of the invention. (See, e.g., Rossi (1994) Current Biology 4: 469-471.) Ribozymes may cleave mRNA at specific cleavage sites. Alternatively, ribozymes may cleave mRNAs at locations dictated by flanking regions that form complementary base pairs with the target mRNA. The construction and production of ribozymes is well known in the art and is described in Meyers (supra).

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. Alternatively, nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases may be included.

Further, an antagonist or antibody that specifically binds the protein or peptide encoded by the polynucleotide may be administered to a subject to treat a disease associated with matrix-remodeling. The antagonist, antibody, or fragment may be used directly to inhibit the activity of the protein or indirectly to deliver a therapeutic agent to cells or tissues which express the protein. The therapeutic agent may be a cytotoxic agent selected from a group including, but not limited to, abrin, ricin, doxorubicin, daunorubicin, taxol, ethidium bromide, mitomycin, etoposide, tenoposide, vincristine, vinblastine, colchicine, dihydroxy anthracin dione, actinomycin D, diphtheria toxin, Pseudomonas exotoxin A and 40, radioisotopes, and glucocorticoid.

Antibodies may be generated using methods that are well known in the art. Such antibodies may include, but are not limited to, polyclonal, monoclonal, chimeric, and single chain antibodies, Fab fragments, and fragments produced by a Fab expression library. Neutralizing antibodies such as those which inhibit dimer formation are especially preferred for therapeutic use. Monoclonal antibodies to the protein may be prepared using any technique which provides for the production of antibody molecules by continuous cell lines in culture. These include, but are not limited to, the hybridoma technique, the human B-cell hybridoma technique, and the EBV-hybridoma technique. In addition, techniques developed for the production of chimeric antibodies can be used (Meyers supra). Alternatively, techniques described for the production of single chain, antibody fragment, or chimeric antibodies which specifically bind the protein or peptide can be used (Pound (1998) Immunochemical Protocols, Methods Mol Biol Vol. 80). Various immunoassays may be used to identify antibodies having the desired specificity. Numerous protocols for competitive binding or immunoradiometric assays using either polyclonal or monoclonal antibodies with established binding specificities are well known in the art.

Yet further, an agonist of a protein may be administered to a subject to treat a matrix remodeling disease which is associated with decreased expression or activity of the protein.

An additional aspect of the invention relates to the administration of a pharmaceutical or sterile composition for any of the therapeutic applications discussed above. Such pharmaceutical compositions may consist of a protein or antibodies, mimetics, agonists, antagonists, or inhibitors of the protein. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a subject alone, or in combination with other agents, drugs, or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing, Easton Pa.).

For any compound, the therapeutically effective dose can be estimated initially either in cell culture assays, or in animal models such as mice, rats, rabbits, dogs, or pigs. An animal model may also be used to determine the concentration range and route of administration. Such information can then be used to determine useful doses and routes for administration in humans.

A therapeutically effective dose refers to that amount of active ingredient which ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population) statistics. Any of the therapeutic methods described above may be applied to any subject in need of such therapy, including, but not limited to, mammals such as dogs, cats, cows, horses, rabbits, monkeys, and most preferably, humans.

Stem Cells and Their Use

SEQ ID NOs:1-20 may be useful in the differentiation of stem cells. Eukaryotic stem cells are able to differentiate into the multiple cell types of various tissues and organs and to play roles in embryogenesis and adult tissue regeneration (Gearhart (1998) Science 282:1061-1062; Watt and Hogan (2000) Science 287:1427-1430). Depending on their source and developmental stage, stem cells may be totipotent with the potential to create every cell type in an organism and to generate a new organism, pluripotent with the potential to give rise to most cell types and tissues, but not a whole organism; or multipotent cells with the potential to differentiate into a limited number of cell types. Stem cells may be transfected with polynucleotides which may be transiently expressed or may be integrated within the cell as transgenes.

Embryonic stem (ES) cell lines are derived from the inner cell masses of human blastocysts and are pluripotent (Thomson et al. (1998) Science 282:1145-1147). They have normal karyotypes and express high levels of telomerase which prevents senescence and allows the cells to replicate indefinitely. ES cells produce derivatives that give rise to embryonic epidermal, mesodermal and endodermal cells. Embryonic germ (EG) cell lines, which are produced from primordial germ cells isolated from gonadal ridges and mesenteries, also show stem cell behavior (Shamblott et al. (1998) Proc Natl Acad Sci 95:13726-13731). EG cells have normal karyotypes and appear to be pluripotent.

Organ-specific adult stem cells differentiate into the cell types of the tissues from which they were isolated. They maintain their original tissues by replacing cells destroyed from disease or injury. Adult stem cells are multipotent and under proper stimulation can be used to generate cell types of various other tissues (Vogel (2000) Science 287:1418-1419). Hematopoietic stem cells from bone marrow provide not only blood and immune cells, but can also be induced to transdifferentiate to form brain, liver, heart, skeletal muscle and smooth muscle cells. Similarly mesenchymal stem cells can be used to produce bone marrow, cartilage, muscle cells, and some neuron-like cells, and stem cells from muscle have the ability to differentiate into muscle and blood cells (Jackson et al. (1999) Proc Natl Acad Sci 96:14482-14486). Neural stem cells, which produce neurons and glia, may also be induced to differentiate into heart, muscle, liver, intestine, and blood cells (Kuhn and Svendsen (1999) BioEssays 21:625-630); Clarke et al. (2000) Science 288:1660-1663; Gage (2000) Science 287:1433-1438; and Galli et al. (2000) Nature Neurosci 3:986-991).

Neural stem cells may be used to treat neurological disorders such as Alzheimer disease, Parkinson disease, and multiple sclerosis and to repair tissue damaged by strokes and spinal cord injuries. Hematopoietic stem cells may be used to restore immune function in immunodeficient patients or to treat autoimmune disorders by replacing autoreactive immune cells with normal cells to treat diseases such as multiple sclerosis, scleroderma, rheumatoid arthritis, and systemic lupus erythematosus. Mesenchymal stem cells may be used to repair tendons or to regenerate cartilage to treat arthritis. Liver stem cells may be used to repair liver damage. Pancreatic stem cells may be used to replace islet cells to treat diabetes. Muscle stem cells may be used to regenerate muscle to treat muscular dystrophies (Fontes and Thomson (1999) BMJ 319:1-3; Weissman (2000) Science 287:1442-1446; Marshall (2000) Science 287:1419-1421; Marmont (2000) Ann Rev Med 51:115-134).

### EXAMPLES

It is understood that this invention is not limited to the particular methodology, protocols, and reagents described, as these may vary. It is also understood that the terminology used herein is for the purpose of describing particular embodiments only and is not intended to limit the scope of the present invention which will be limited only by the appended claims. The examples below are provide to illustrate the subject invention and are not included for the purpose of limiting the invention.

I cDNA Library Construction

The cDNA library, THYMFET02, was selected to demonstrate the construction of the cDNA libraries from which novel matrix-remodeling polynucleotides were derived. The THYMFET02 cDNA library was constructed from microscopically normal thymus tissue obtained from a Caucasian female fetus who died at 17 weeks gestation from anencephaly. Serology was negative; family history included tobacco abuse and gastritis.

The frozen tissue was homogenized and lysed in TRIZOL reagent (1 gm tissue/10 ml; Life Technologies, Rockville Md.), using a POLYTRON homogenizer (Brinkmann Instruments, Westbury N.Y.). After a brief incubation on ice, chloroform was added (1:5 v/v), and the lysate was centrifuged. The upper chloroform layer was removed, and the RNA was precipitated with isopropanol, resuspended in DEPC-treated water, and treated with DNAse for 25 min at 37° C.

The mRNA was extracted again with acid phenol-chloroform, pH 4.7, and precipitated using 0.3 M sodium acetate and 2.5 volumes ethanol. The mRNA was isolated using the OLIGOTEX kit (Qiagen, Chatsworth Calif.) and used to construct the cDNA library.

The MRNA was handled according to the recommended protocols in the SUPERSCRIPT plasmid system (Life Technologies). The cDNAs were fractionated on a SEPHAROSE CL4B column (Amersham Pharmacia Biotech, Piscataway N.J.), and those cDNAs exceeding 400 bp were ligated into pINCY plasmid (Incyte Genomics, Palo Alto Calif.). The plasmid was subsequently transformed into DH5α competent cells (Life Technologies).

II Isolation and Sequencing of cDNA Clones

Plasmid DNA was released from the cells and purified using the REAL PREP 96 plasmid kit (Qiagen). This kit enabled the simultaneous purification of 96 samples in a 96-well block using multi-channel reagent dispensers. The recommended protocol was employed except for the following changes: 1) the bacteria were cultured in 1 ml of sterile TERRIFIC BROTH (BD Biosciences Sparks Md.) with carbenicillin (Carb) at 25 mg/l and glycerol at 0.4%; 2) after inoculation, the cultures were incubated for 19 hours and at the end of incubation, the cells were lysed with 0.3 ml of lysis buffer; and 3) following isopropanol precipitation, the plasmid DNA pellet was resuspended in 0.1 ml of distilled water. After the last step in the protocol, samples were transferred to a 96-well block for storage at 4° C.

The cDNAs were prepared using a MICROLAB 2200 system (Hamilton, Reno Nev.) in combination with DNA ENGINE thermal cyclers (MJ Research, Watertown Mass.) and sequenced by the method of Sanger and Coulson (1975, J Mol Biol 94:441f) using ABI PRISM 377 DNA sequencing systems (ABI).

III Selection, Assembly, and Characterization of Sequences

The sequences used for coexpression analysis were assembled from EST sequences, 5′ and 3′ longread sequences, and full length coding sequences. Selected assembled sequences were expressed in at least three cDNA libraries.

The assembly process is described as follows. EST sequence chromatograms were processed and verified. Quality scores were obtained using PHRED (Ewing et al. (1998) Genome Res 8:175-185; Ewing and Green (1998) Genome Res 8:186-194). Then the edited sequences were loaded into a relational database management system (RDBMS). The EST sequences were clustered into an initial set of bins using BLAST with a product score of 50. All clusters of two or more sequences were created as bins. The overlapping sequences represented in a bin correspond to the sequence of a transcribed gene.

Assembly of the component sequences within each bin was performed using a modification of PHRAP, a publicly available program for assembling DNA fragments (Phil Green, University of Washington, Seattle Wash.). Bins that showed 82% identity from a local pair-wise alignment between any of the consensus sequences were merged.

Bins were annotated by screening the consensus sequence in each bin against public databases, such as GBpri and GenPept from NCBI. The annotation process involved a FASTn screen against the GBpri database in GenBank. Those hits with a percent identity of greater than or equal to 70% and an alignment length of greater than or equal to 100 base pairs were recorded as homolog hits. The residual unannotated sequences were screened by FASTx against GenPept. Those hits with an E value of less than or equal to 10⁻⁸ are recorded as homolog hits.

Sequences were then reclustered using BLASTn and CROSS-MATCH, a program for rapid protein and nucleic acid sequence comparison and database search (Green, supra), sequentially. Any BLAST alignment between a sequence and a consensus sequence with a score greater than 150 was realigned using CROSS-MATCH. The sequence was added to the bin whose consensus sequence gave the highest Smith-Waterman score amongst local alignments with at least 82% identity. Non-matching sequences created new bins. The assembly and consensus generation processes were performed for the new bins.

IV Coexpression Analyses of Known Matrix-remodeling Genes

Twenty one known matrix-remodeling genes were selected to identify novel genes that are closely associated with matrix-remodeling. The known genes were BM-40, C/DSPG, collagen I, II, II, and IV, CTGF, fibrillin, fibronectins, fibr-r, fibulin 1, HSPG, hevin, IGF 1, IGFBP, laminin, lumican, MGP, MMPs, TIMP 1, 2, and 3. The protein products of the known matrix-remodeling genes may be categorized as follows.

1. Extracellular matrix component protein. These proteins include collagens, proteoglycans, fibrillin, fibronectin, fibulin, and laminin that constitute the major structures of the extracellular matrix.

2. Matrix proteases and matrix protease inhibitors. These proteins include matrix metalloproteases (MMPs) such as the collagenases, and MMP inhibitors such as the tissue-inhibitors of matrix metalloproteases (TIMPs).

3. Regulatory proteins that control expression of matrix-remodeling genes. Such regulatory proteins include connective tissue growth factor, insulin-like growth factor, osteonectin (BM-40), and the receptors for and inhibitors of these proteins.

The known matrix-remodeling genes that we examined in this analysis, and brief descriptions of their functions, are listed below. Detailed descriptions of their roles in matrix-remodeling may be found in the cited articles and reviews, incorporated by reference herein.

| Gene | Description and References |
|---|---|
| BM-40 | Alternate names: SPARC, osteonectin |
| | Regulates connective tissue remodeling, wound healing, |
| | angiogenesis |
| | Induces matrix metalloprotease synthesis |
| | (collagenase & gelatinase) |
| | Regulates cell movement and proliferation |
| | Expression increased in neoplastic melanoma, fibrosis, |
| | angiogenesis. |
| | (Kamihagi et al. (1994) Biochem Biophys |
| | Res Commun 200:423-8; Lane et al. (1994) J Cell Biol |
| | 125:929-43; Inagaki et al. (1996) Life Sci 58:927-34; |
| | Ledda et al. (1997) J Invest Dermatol 108:210-4; |
| | Shankavaram et al. (1997) J Cell Physiol 173:327-34) |
| C/DSPG | Chondroitin/dermatan sulfate proteoglycans |
| | Major extracellular matrix proteoglycan |
| | Regulate cell proliferation, attachment and migration |
| | (Darnell et al. (1990) Molecular Cell Biology, |
| | Scientific American Books, New York NY; Toole (1991) |
| | In: Cell Biology of Extracellular Matrix, |
| | Plenum, New York NY, pp. 305-341; Beck et al. |
| | (1993) Biochem Biophys Res Commun 190:616-23) |
| Collagens | Family of fibrous structural proteins |
| | (collagen I, II, III, IV, etc.) |
| | Most abundant structural component of the extracellular |
| | matrix |
| | Secreted as procollagen; converted to collagen by MMPs |
| | (Alexander and Werb (1991) In: |
| | Cell Biology of Extracellular Matrix, pp. |
| | 255-302 supra; Adams (1993) In: |
| | Extracellular Matrix, Marcel Dekker, New |
| | York NY pp. 91-119; Schuppan et al. (1993) |
| | In: Extracellular Matrix, pp. 201-254, supra) |
| CTGF | Connective tissue growth factor |
| | Mediates induction of matrix synthesis and fibrosis |
| | (Grotendorst (1997) Cytokine Growth Factor Rev 8:171-9; |
| | Oemar and Luscher (1997) Arterioscler Thromb Vasc |
| | Biol 17:1483-9; Ito et al. |
| | (1998) Kidney Int 53:853-61) |
| fibrillin | Major component of extracellular microfibrills |
| | (matrix elastic network) |
| | Present in connective tissue throughout the body |
| | (Kielty and Shuttleworth (1995) Int J Biochem Cell |
| | Biol 27:747-60; Haynes et al. |
| | (1997) Br J Dermatol 137:17-23; Hayward and Brock |
| | (1997) Hum Mutat 10:415-23) |
| fibronectins | Family of extracellular matrix glycoproteins |
| | Anchor cells to the matrix |
| | Bind matrix proteins to cell surface receptors |
| fibr-r | Fibronectin receptor |
| | Fibronectin receptors regulate cell adhesion & migration |
| | (Darnell supra; Ruoslahti (1991) |
| | Cell Biology of Extracellular Matrix, |
| | pp. 343-363 supra; Yamada (1991) |
| | Cell Biology of Extracellular Matrix, |
| | pp. 111-146, supra) |
| fibulin 1 | Fibronectin-binding extracellular matrix protein |
| | Mediates platelet adhesion via a bridge of fibrinogen |
| | Cleaved by matrix metalloproteinases |
| | Inhibits breast and ovarian cancer cell motility |
| | (Argraves et al. (1990) J Cell Biol 111:3155-64; |
| | Sasaki et al. (1996) Eur J Biochem 240:427-34; |
| | Hayashido et al. (1998) Int J Cancer 75:654-8) |
| HSPG | Heparan sulfate proteoglycans |
| | Extracellular matrix proteoglycan found on |
| | cell surface of many cell types |
| | Regulate cell interactions with the extracellular matrix |
| | Bind to collagens and fibronectin in the matrix |
| | Regulate cell proliferation, attachment and migration |
| | (Darnell (supra); Toole (supra); Schuppan (supra) |
| hevin | Extracellular matrix protein |
| | Homolog to BM-40 |
| | Regulates cell adhesion and migration |
| | Downregulated in metastatic prostate cancer, lung cancer |
| | (Girard and Springer (1996) J Biol Chem 271:4511-7; |
| | Bendik et al. Cancer Res 58:232-6) |
| IGF 1 | Insulin-like growth factor |
| | Regulates matrix homeostasis and remodeling |
| | Regulates aggregation, growth and survival of cancer cells |
| | (Aston et al. (1995) Am J Respir Crit Care Med |
| | 151:1597-603; Bitar and Labbad (1996) J Surg Res |
| | 61:113-9; Guvakova and Surmacz (1997) Exp Cell |
| | Res 231:149-62; |
| | Sunic et al. (1998) Endocrinology 139:2356-62) |
| IGFBP | Insulin-like growth factor binding protein |
| | Regulates IGF-1 bioavailability (binds IGF-1 more strongly |
| | than the receptor) |
| | Degraded by matrix metalloproteases |
| | (Kiefer et al. (1991) Biochem Biophys Res Commun |
| | 176:219-25; Fowlkes et al. (1995) Prog Growth Factor |
| | Res 6:255-63; Parker et al. (1996) J Biol |
| | Chem 271:13523-9) |
| laminin | Maj or protein in basal lamina, with collagen, |
| | HSPG, and entactin |
| | Anchors cells to the matrix by binding collagen, |
| | HSGP and heparin |
| | Laminins and collagens are the main targets of MMPs |
| | Regulates cell attachment, migration, growth, |
| | and differentiation |
| | (Yamada et al. (1993) In: Extracellular Matrix, |
| | pp. 49-66 (supra); Giannelli et al. |
| | (1997) Science 277:225-8; Quaranta and Plopper (1997) |
| | Kidney Int 51:1441-6; Soini et al. |
| | (1997) Hum Pathol 28:220-6) |
| lumican | Extracellular proteoglycan |
| | Organizes collagen fibrils in extracellular matrix |
| | (Dourado et al. (1996) Osteoarthritis Cartilage 4:187-96; |
| | Scott (1996) Bio-chemistry 35:8795-9; Cs-Szabo et al. |
| | (1997) Arthritis Rheum 40:1037-45) |
| MGP | Matrix Gla protein |
| | Regulates calcification of cartilage |
| | Marker for osteoblast activity |
| | (Shanahan et al. (1994) J Clin Invest 93:2393-402; |
| | Luo et al. (1997) Nature 386:78-81; |
| | Martinetti et al. (1997) Tumour Biol 18:197-205) |
| MMP | Family of Matrix Metalloproteases (including collagenases) |
| | Cleave procollagen to produce collagen (Alexander and |
| | Werb (1991) In: Cell Biology of Extracellular Matrix, |
| | pp. 255-302; Adams (supra); Schuppan |
| TIMP 1, 2, 3 | Tissue inhibitors of matrix metalloproteinases |
| | Bind and inactivate matrix proteases |
| | (Schuppan (supra); Zvibel and Kraft (1993) |
| | In: Extracellular Matrix, pp. 559-580) |

The coexpression of the 21 known genes with each other is shown below in Table 3. Entries are the negative log of the p-value (−log p) for the coexpression of any two genes. As shown, the method successfully identified the strong associations among the known genes which indicates that the coexpression analysis method of the present invention was effective in identifying genes that are closely associated with matrix-remodeling.

TABLE 3
Coexpression of 21 known matrix-remodeling genes. (−log p)

| | laminin | fibrillin | lumican | coll IV | TIMP-1 | IGFBP | coll VI | TIMP-3 | CTGF | hevin | fibulin |
|---|---|---|---|---|---|---|---|---|---|---|---|
| laminin | | 7 | 9 | 21 | 9 | 15 | 8 | 4 | 5 | 7 | 14 |
| fibrillin | 7 | | 13 | 8 | 6 | 7 | 14 | 11 | 4 | 7 | 12 |
| lumican | 9 | 13 | | 24 | 17 | 16 | 28 | 17 | 17 | 14 | 15 |
| coll IV | 21 | 8 | 24 | | 17 | 22 | 22 | 13 | 11 | 14 | 28 |
| TIMP-1 | 9 | 6 | 17 | 17 | | 20 | 15 | 11 | 11 | 6 | 10 |
| IGFBP | 15 | 7 | 16 | 22 | 20 | | 20 | 18 | 16 | 11 | 14 |
| coll VI | 8 | 14 | 28 | 22 | 15 | 20 | | 13 | 17 | 19 | 16 |
| TIMP-3 | 4 | 11 | 17 | 13 | 11 | 18 | 13 | | 13 | 18 | 20 |
| CTGF | 5 | 4 | 17 | 11 | 11 | 16 | 17 | 13 | | 8 | 10 |
| hevin | 7 | 7 | 14 | 14 | 6 | 11 | 19 | 18 | 8 | | 15 |
| fibulin | 14 | 12 | 15 | 28 | 10 | 14 | 16 | 20 | 10 | 15 | |
| BM-40 | 10 | 7 | 22 | 25 | 21 | 18 | 20 | 22 | 18 | 18 | 19 |
| TIMP-2 | 7 | 8 | 10 | 12 | 15 | 14 | 11 | 14 | 7 | 13 | 9 |
| HSPG | 11 | 4 | 8 | 22 | 9 | 19 | 11 | 9 | 7 | 8 | 11 |
| fibronectin | 9 | 8 | 12 | 16 | 16 | 21 | 19 | 10 | 19 | 8 | 8 |
| MGP | 19 | 6 | 25 | 27 | 20 | 25 | 19 | 18 | 22 | 23 | 19 |
| C/DSPG | 11 | 13 | 33 | 26 | 13 | 23 | 28 | 25 | 12 | 27 | 20 |
| fibr-r | 7 | 6 | 14 | 12 | 8 | 10 | 12 | 12 | 12 | 10 | 6 |
| coll-I | 16 | 11 | 32 | 34 | 14 | 27 | 31 | 12 | 18 | 14 | 17 |
| coll-III | 10 | 12 | 34 | 25 | 20 | 23 | 36 | 13 | 13 | 11 | 20 |
| MMP | 13 | 11 | 17 | 26 | 19 | 20 | 27 | 9 | 11 | 8 | 18 |

| | BM-40 | TIMP-2 | HSPG | fibronectin | MGP | C/DSPG | fibr-r | coll-I | coll-III | MMP |
|---|---|---|---|---|---|---|---|---|---|---|
| laminin | 10 | 7 | 11 | 9 | 19 | 11 | 7 | 16 | 10 | 13 |
| fibrillin | 7 | 8 | 4 | 8 | 6 | 13 | 6 | 11 | 12 | 11 |
| lumican | 22 | 10 | 8 | 12 | 25 | 33 | 14 | 32 | 34 | 17 |
| coll IV | 25 | 12 | 22 | 16 | 27 | 26 | 12 | 34 | 25 | 26 |
| TIMP-1 | 21 | 15 | 9 | 16 | 20 | 13 | 8 | 14 | 20 | 19 |
| IGFBP | 18 | 14 | 19 | 21 | 25 | 23 | 10 | 27 | 23 | 20 |
| coll VI | 20 | 11 | 11 | 19 | 19 | 28 | 12 | 31 | 36 | 27 |
| TIMP-3 | 22 | 14 | 9 | 10 | 18 | 25 | 12 | 12 | 13 | 9 |
| CTGF | 18 | 7 | 7 | 19 | 22 | 12 | 12 | 18 | 13 | 11 |
| hevin | 18 | 13 | 8 | 8 | 23 | 27 | 10 | 14 | 11 | 8 |
| fibulin | 19 | 9 | 11 | 8 | 19 | 20 | 6 | 17 | 20 | 18 |
| BM-40 | | 14 | 11 | 24 | 21 | 24 | 16 | 25 | 32 | 19 |
| TIMP-2 | 14 | | 7 | 12 | 8 | 16 | 11 | 13 | 13 | 13 |
| HSPG | 11 | 7 | | 8 | 14 | 10 | 6 | 11 | 10 | 10 |
| fibronectin | 24 | 12 | 8 | | 14 | 14 | 11 | 24 | 21 | 15 |
| MGP | 21 | 8 | 14 | 14 | | 32 | 14 | 25 | 20 | 13 |
| C/DSPG | 24 | 16 | 10 | 14 | 32 | | 14 | 27 | 28 | 14 |
| fibr-r | 16 | 11 | 6 | 11 | 14 | 14 | | 14 | 13 | 6 |
| coll-I | 25 | 13 | 11 | 24 | 25 | 27 | 14 | | 42 | 21 |
| coll-III | 32 | 13 | 10 | 21 | 20 | 28 | 13 | 42 | | 23 |
| MMP | 19 | 13 | 10 | 15 | 13 | 14 | 6 | 21 | 23 | |

V Novel Polynucleotides Associated with Matrix-remodeling

Using coexpression analysis, 20 novel polynucleotides that show strong association with known matrix-remodeling genes were identified from among a total of 41,419 polynucleotides. The degree of association was measured by probability values and has a cutoff of p value less than 0.00001 (highly significant). This was followed by annotation and literature searches to insure that the genes that passed the probability test have strong association with known matrix-remodeling genes. This process was reiterated so that the initial 41,419 polynucleotides were reduced to the final 20 matrix-remodeling polynucleotides. Details of the coexpression patterns for the 20 novel matrix-remodeling polynucleotides are presented below.

Each of the 20 novel polynucleotides is coexpressed with at least two of the 21 known matrix-remodeling genes with a p-value of less than 10⁻⁷. The coexpression results are shown in Table 4 below. The novel polynucleotides are listed in the table by their Incyte clone numbers (Clone), and the known genes by their abbreviated names as shown in Example IV.

TABLE 4
Coexpression of 20 Polynucleotides with Known Matrix-remodeling Genes. (−log p)

| Gene | | | | | | | | | | | |
|---|---|---|---|---|---|---|---|---|---|---|---|
| Clone | laminin | fibrillin | lumican | coll IV | TIMP-1 | IGFBP | coll VI | TIMP-3 | CTGF | hevin | fibulin |
| 606132 | 8 | 7 | 2 | 6 | 4 | 7 | 7 | 2 | 4 | 4 | 4 |
| 627722 | 3 | 4 | 1 | 1 | 3 | 3 | 2 | 5 | 3 | 6 | 3 |
| 639644 | 6 | 7 | 11 | 10 | 3 | 4 | 7 | 3 | 14 | 6 | 6 |
| 1362659 | 6 | 5 | 6 | 7 | 6 | 9 | 10 | 9 | 8 | 8 | 7 |
| 1446685 | 6 | 6 | 11 | 13 | 4 | 7 | 8 | 5 | 7 | 5 | 10 |
| 1556751 | 3 | 7 | 7 | 8 | 8 | 9 | 9 | 8 | 7 | 6 | 5 |
| 1656953 | 6 | 8 | 6 | 2 | 5 | 7 | 8 | 5 | 6 | 9 | 3 |
| 1662318 | 9 | 3 | 6 | 10 | 7 | 9 | 5 | 5 | 8 | 8 | 6 |
| 1996726 | 3 | 4 | 7 | 7 | 6 | 5 | 8 | 3 | 10 | 2 | 2 |
| 2137155 | 3 | 2 | 6 | 3 | 4 | 2 | 2 | 4 | 6 | 4 | 2 |
| 2268890 | 9 | 13 | 7 | 9 | 8 | 11 | 8 | 9 | 5 | 5 | 8 |
| 2305981 | 3 | 2 | 4 | 6 | 3 | 4 | 3 | 5 | 5 | 6 | 7 |
| 2457612 | 3 | 3 | 3 | 5 | 2 | 4 | 4 | 2 | 8 | 4 | 5 |
| 2814981 | 6 | 3 | 5 | 7 | 4 | 6 | 7 | 2 | 2 | 5 | 5 |
| 3089150 | 4 | 6 | 11 | 8 | 5 | 10 | 13 | 9 | 14 | 10 | 11 |
| 3206667 | 8 | 5 | 10 | 9 | 7 | 5 | 6 | 4 | 9 | 4 | 7 |
| 3284695 | 7 | 6 | 7 | 14 | 8 | 7 | 6 | 14 | 8 | 18 | 12 |
| 3481610 | 3 | 2 | 4 | 4 | 3 | 6 | 4 | 6 | 6 | 7 | 4 |
| 3722004 | 6 | 4 | 8 | 10 | 13 | 9 | 7 | 13 | 8 | 9 | 11 |
| 3948614 | 11 | 8 | 6 | 17 | 8 | 13 | 12 | 5 | 5 | 11 | 12 |

| Gene | | | | | | | | | | |
|---|---|---|---|---|---|---|---|---|---|---|
| Clone | BM-40 | TIMP-2 | HSPG | fibronectin | MGP | C/DSPG | fibr-r | coll-I | coll-III | MMP |
| 606132 | 3 | 3 | 4 | 4 | 3 | 2 | 2 | 5 | 3 | 10 |
| 627722 | 4 | 3 | 2 | 6 | 5 | 3 | 3 | 2 | 3 | 4 |
| 639644 | 9 | 6 | 2 | 9 | 8 | 5 | 6 | 9 | 7 | 6 |
| 1362659 | 6 | 8 | 6 | 7 | 9 | 9 | 7 | 10 | 5 | 5 |
| 1446685 | 9 | 5 | 9 | 5 | 9 | 8 | 6 | 8 | 10 | 7 |
| 1556751 | 5 | 7 | 8 | 4 | 10 | 11 | 3 | 7 | 6 | 8 |
| 1656953 | 7 | 4 | 3 | 4 | 10 | 8 | 7 | 4 | 4 | 5 |
| 1662318 | 8 | 5 | 9 | 6 | 8 | 6 | 4 | 7 | 7 | 9 |
| 1996726 | 3 | 2 | 2 | 9 | 3 | 6 | 6 | 8 | 11 | 6 |
| 2137155 | 9 | 4 | 2 | 8 | 4 | 4 | 4 | 5 | 2 | 5 |
| 2268890 | 7 | 8 | 5 | 8 | 8 | 11 | 3 | 11 | 7 | 11 |
| 2305981 | 5 | 2 | 2 | 2 | 7 | 6 | 4 | 3 | 2 | 2 |
| 2457612 | 5 | 2 | 2 | 7 | 8 | 6 | 6 | 5 | 4 | 8 |
| 2814981 | 5 | 3 | 6 | 5 | 4 | 6 | 1 | 6 | 4 | 7 |
| 3089150 | 10 | 7 | 6 | 8 | 11 | 16 | 11 | 9 | 7 | 5 |
| 3206667 | 8 | 4 | 4 | 7 | 13 | 12 | 4 | 8 | 8 | 6 |
| 3284695 | 9 | 10 | 8 | 6 | 18 | 10 | 5 | 13 | 6 | 6 |
| 3481610 | 5 | 1 | 5 | 5 | 7 | 5 | 3 | 3 | 2 | 2 |
| 3722004 | 12 | 11 | 5 | 10 | 9 | 12 | 3 | 7 | 7 | 6 |
| 3948614 | 7 | 11 | 13 | 4 | 7 | 7 | 4 | 14 | 11 | 10 |

VI Description of the Polynucleotides Associated with Matrix-remodeling

The 20 novel polynucleotides were identified from the data shown in Table 4 to be associated with matrix-remodeling. The nucleic acid sequences comprising the consensus sequences of SEQ ID NOs:1-20 of the present invention were first identified from Incyte Clones 606132, 627722, 639644, 1362659, 1446685, 1556751, 1656953, 1662318, 1996726, 2137155, 2268890, 2305981, 2457612, 2814981, 3089150, 3206667, 3284695, 3481610, 3722004, and 3948614, respectively, and assembled according to Example III. BLAST was performed for SEQ ID NOs:1-20 according to Example VII. The sequences of SEQ ID NOs:1-20 were translated, and the translations were compared with known motifs as described in Example VII. Proteins comprising the amino acid sequences of SEQ ID NO:21, SEQ ID NO:22, and SEQ ID NO:23 of the present invention were encoded by SEQ ID NO:2, SEQ ID NO:6, and SEQ ID NO:11, respectively. Translation of SEQ ID NO:2, SEQ ID NO:6, and SEQ ID NO:11 are shown in FIGS. 1, 2 and 3, respectively. SEQ ID NOs:21-23 were analyzed using BLAST and other motif search tools as disclosed in Example VII.

SEQ ID NO:3 is 2987 residues in length and shows about 59% sequence identity from about nucleotide 2117 to about nucleotide 2914 with the cDNA encoding regulatory subunit of a human cAMP-dependent protein kinase, RIIbeta (WO 88/03164). As can be seen in Table 4 above, it is most highly co-expressed with CTGF (p-value=14) and highly expressed with lumican (p-value=11) and collagen IV (p-value=10). FIGS. 4 and 5 which show cell, tissue and system specific expression and the differential expression of SEQ ID NO:3 in pancreatic tumor, respectively, were produced using the LIFESEQ Gold database (Incyte Genomics). FIGS. 4 and 5 serve as examples of the data present in LIFESEQ Gold from which the p-values for each of the claimed sequences of Table 4 were derived.

SEQ ID NO:8 is 3017 nucleotides in length and shows about 70% to about 74% sequence identity from about nucleotide 1 to about nucleotide 1260 and about nucleotide 1925 to about nucleotide 1985 with human Hpast mRNA (g2529706), a gene associated with multiple endocrine neoplasia type 1.

SEQ ID NO:9 is 1735 nucleotides in length and shows about 25% sequence identity from about nucleotide 5 to about nucleotide 1534 with a human neuronal cell adhesion molecule (WO 96/04396) important in the development of nervous system by promoting cell-cell adhesion.

SEQ ID NO:14 is 2040 nucleotides in length and shows about 60% to 70% sequence identity from about nucleotide 1 to about nucleotide 1023 with a human mRNA for a serine protease (g1621243) specific for insulin-like growth factor-binding proteins. The amino acid sequence encoded by SEQ ID NO:14 from about nucleotide 3 to about nucleotide 1043 shows about 61% sequence identity with an osteoblast-like cell-derived protein (J09107980) useful for treatment and prevention of various diseases and as contraceptive.

SEQ ID NO:15 is 2121 nucleotides in length and shows 60-80% sequence identity with a mouse gene, ADAMT-1 (g2809056), a member of the ADAM (the disintegrin and metalloproteinase) family. ADAMT-1 has been shown to contain the thrombospondin (TSP) type I motif; expression of ADAMT-1 is closely associated with inflammatory processes (Kuno et al (1997) Genomics 46:466-471).

SEQ ID NO:16 is 2900 nucleotides in length and shows about 70% sequence identity with a mouse homeobox (Pmx) mRNA (g460124). Homeobox genes are expressed in very specific temporal and spatial pattern and function as transcriptional regulators of developmental processes (Kern et al. (1994) Genomics 19:334-340).

SEQ ID NO:21 is 551 amino acid residues long and shows about 37% sequence identity from about amino acid residue 10 to about amino acid residue 278 with PALM (g3219602), a human paralemin that is membrane-bound and expressed abundantly in brain and at intermediate levels in the kidney and in endocrine cells. In addition, the sequence encompassing residues 418 to 434 of SEQ ID NO:21 resembles one of the structural fingerprint regions of a seven trans-membrane receptor, LCR1, that is isolated from the human brain (Rimland et al. (1991) Mol Pharmacol 40:869-875). SEQ ID NO:21 also has one potential amidation site at L546; three potential N-glycosylation sites at N223, N229, and N408; one potential cAMP- and cGMP-dependent protein kinase phosphorylation site at S486; fifteen potential casein kinase II phosphorylation sites at S57, S100, T101, T116, S135, S253, T349, S370, T387, S426, T434, S489, S505, S520, and T526; one potential N-myristoylation site at G54; and nine potential protein kinase C phosphorylation sites at T15, S25, S57, S100, S123, S247, S364, S370, and S505.

SEQ ID NO:22 is 99 amino acid residues in length. The sequence of SEQ ID NO:22 from about amino acid residue 71 to about amino acid residue 81 resembles one of the fingerprint regions of the RH1 and RH2 opsins, a family of G protein coupled receptors that mediate vision (Zuker et al. (1985) Cell 40:851-858; Cowman et al. (1986) Cell 44:705-710). SEQ ID NO:22 also has one potential N-myristoylation site at G24, and two potential protein kinase C phosphorylation sites at S13 and S89.

SEQ ID NO:23 is 493 amino acid residues in length and shows about 44% sequence identity from about amino acid residue 277 to about amino acid residue 487 with an angiopoietin-like factor from the human cornea, CDT6 (g2765527). Angiopoietin 1 and angiopoietin 2 function as a natural ligand and a natural inhibitor, respectively, for TIE2, a receptor critical in angiogenesis during embryonic development, tumor growth, and tumor metastasis. The sequences encompassing amino acid residues 305 to 343, 346 to 355, 365 to 402, 411 to 424, and 428 to 458 of SEQ ID NO:23 resemble the carboxy-terminal domain signatures of fibrinogen beta and gamma chains from BLOCKS analysis. SEQ ID NO:23 also exhibits one potential signal peptide region encompassing amino acid residues M1 to G22 when analyzed using a HMM-based signal peptide analysis tool. In addition, SEQ ID NO:23 shows two potential N-glycosylation sites at N164 and N192; one potential cAMP- and cGMP-dependent protein kinase phosphorylation sites at S 127, six potential casein kinase II phosphorylation sites at S34, S209, T238, S266, T368, and T417; four potential N-myristoylation sites at G12, G18, G22, and G29; eight potential protein kinase C phosphorylation sites at S34, S209, T268, T299, T335, S373, S383, and S477; and three potential tyrosine kinase phosphorylation sites at Y183, Y392, and Y467.

VII Homology Searching of the Polynucleotides and Their Encoded Proteins

Polynucleotides, SEQ ID NOs:1-20, and proteins, SEQ ID NOs:21-23, were queried against databases derived from sources such as GenBank and SwissProt. These databases, which contain previously identified and annotated sequences, were searched for regions of similarity using BLAST and Smith-Waterman alignment (Smith et al. (1992) Protein Engineering 5:35-51). BLAST searched for matches and reported only those that satisfied the probability thresholds of 10⁻²⁵ or less for polynucleotide sequences and 10⁻⁸ or less for protein sequences.

The proteins were also analyzed for known motif patterns using MOTIFS, SPSCAN, BLIMPS, and Hidden Markov Model (HMM)-based protocols. MOTIFS (Genetics Computer Group, Madison Wis.) searches protein sequences for patterns that match those defined in the Prosite Dictionary of Protein Sites and Patterns (Bairoch et al. supra), and displays the patterns found and their corresponding literature abstracts. SPSCAN (Genetics Computer Group) searches for potential signal peptide sequences using a weighted matrix method (Nielsen et al. (1997) Prot Eng 10:1-6). Hits with a score of 5 or greater were considered. BLIMPS uses a weighted matrix analysis algorithm to search for sequence similarity between the amino acid sequences and those contained in BLOCKS, a database consisting of short amino acid segments, or blocks, of 3-60 amino acids in length, compiled from the PROSITE database (Henikoff and Henikoff supra; Bairoch et al. supra), and those in PRINTS, a protein fingerprint database based on non-redundant sequences obtained from sources such as SwissProt, GenBank, PIR, and NRL-3D (Attwood et al. (1997) J Chem Inf Comput Sci 37:417-424). For the purposes of the present invention, the BLIMPS searches reported matches with a cutoff score of 1000 or greater and a cutoff probability value of 1.0×10⁻³. HMM-based protocols were based on a probabilistic approach and searched for consensus primary structures of gene families in the protein sequences (Eddy, supra; Sonnhammer, supra). More than 500 known protein families with cutoff scores ranging from 10 to 50 bits were selected for use in this invention.

VIII Labeling and Use of Individual Hybridization Probes

Oligonucleotides are designed using state-of-the-art software such as OLIGO primer analysis software (Molecular Biology Insights) and labeled by combining 50 pmol of each oligomer, 250 μCi of [γ-³²P] adenosine triphosphate (Amersham Pharmacia Biotech), and T4 polynucleotide kinase (NEN Life Science Products, Boston Mass.). The labeled oligonucleotides are purified using a SEPHADEX G-25 superfine resin column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba 1, or Pvu II (NEN Life Science Products).

The DNA from each digest is fractionated on a 0.7 percent agarose gel and transferred to NYTRAN PLUS membranes (Schleicher & Schuell, Keene N.H.). Hybridization is carried out under the following conditions: 5× SCC/0.1% SDS at 60° C. for about 6 hours, subsequent washes are performed at higher stringency with buffers, such as 1× SCC/0.1% SDS at 45° C., then 0.1× SCC. After XOMAT AR film (Eastman Kodak, Rochester N.Y.) is exposed to the blots for several hours, hybridization patterns are compared.

IX Production of Specific Antibodies

SEQ ID NO:20, 21, or 23 purified using polyacrylamide gel electrophoresis (Harrington (1990) Methods Enzymol 182:488-495), or other purification techniques, is used to immunize rabbits and to produce antibodies using standard protocols.

Alternatively, the protein sequence is analyzed using LASERGENE software (DNASTAR, Madison Wis.) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. Typically, oligopeptides 15 residues in length are synthesized using an ABI 431A peptide synthesizer (Applied Biosystems) using Fmoc-chemistry and coupled to KLH (Sigma-Aldrich, St. Louis Mo.) by reaction with N-maleimidobenzoyl-N-hydroxysuccinimide ester to increase immunogenicity. Rabbits are immunized with the oligopeptide-KLH complex in complete Freund's adjuvant. Resulting antisera are tested for antipeptide activity by, for example, binding the peptide to plastic, blocking with 1% BSA, reacting with rabbit antisera, washing, and reacting with radio-iodinated goat anti-rabbit IgG.

All patents and publications mentioned in the specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A composition comprising a plurality of polynucleotides having the nucleic acid sequences of SEQ ID NOs:1-13 or the complements thereof.

2. An isolated polynucleotide comprising a nucleic acid sequence selected from SEQ ID NOs: 1-20 or the complement thereof.

3. A composition comprising a polynucleotide of claim 2 and a labeling moiety.

4. A method of using a composition to screen a plurality of molecules to identify at least one ligand which specifically binds a polynucleotide of the composition, the method comprising:
a) combining the composition of claim 1 with molecules under conditions to allow specific binding; and
b) detecting specific binding, thereby identifying a ligand which specifically binds the polynucleotide.

5. The method of claim 4 wherein the molecules to be screened are selected from DNA molecules, RNA molecules, peptide nucleic acids, mimetics, and proteins.

6. A method of using a polynucleotide to purify a ligand, the method comprising:
a) combining the polynucleotide of claim 2 with a sample under conditions to allow specific binding;
b) recovering the bound polynucleotide; and
c) separating the ligand from the bound polynucleotide, thereby obtaining purified ligand.

8. The method of claim 7 wherein the polynucleotide is attached to a substrate.

9. The method of claim 7 wherein the molecules to be screened are selected from DNA molecules, RNA molecules, peptide nucleic acids, mimetics, and proteins.

10. A method for using a composition to detect gene expression in a sample containing nucleic acids, the method comprising:
a) hybridizing the composition of claim 1 to the nucleic acids under conditions for formation of one or more hybridization complexes; and
b) detecting hybridization complex formation, wherein complex formation indicates gene expression in the sample.

11. The method of claim 9 wherein the composition is attached to a substrate.

12. The method of claim 9, gene expression indicates the presence of cancer.

13. A vector comprising a polynucleotide of claim 2.

14. A host cell comprising the vector of claim 13.

15. A method for using a host cell to produce a protein, the method comprising:
a) culturing the host cell of claim 14 under conditions for expression of the protein; and
b) recovering the protein from cell culture.

16. A purified protein obtained using the method of claim 15.

17. A composition comprising the protein of claim 16 and a pharmaceutical carrier.

18. A method for using a protein to screen a plurality of molecules to identify at least one ligand which specifically binds the protein, the method comprising:
a) combining the protein of claim 16 with the plurality of molecules under conditions to allow specific binding; and
b) detecting specific binding, thereby identifying a ligand which specifically binds the protein.

19. The method of claim 18 wherein the plurality of molecules is selected from DNA molecules, RNA molecules, peptide nucleic acids, mimetics, proteins, agonists, antagonists, and antibodies.

20. A method of using a protein to purify a ligand from a sample, the method comprising:
a) combining the protein of claim 16 with a sample under conditions to allow specific binding;
b) recovering the bound protein; and
c) separating the ligand from the bound protein, thereby obtaining purified ligand.


SEQUENCE LISTING (23 sequences)

SEQ ID NO: 1 - DNA, 1447 bp, Homo sapiens
  unsure 1380: a or g or c or t, unknown, or other
cctggaaccagaaggagacctacctgcacatcatgaagaacgaggaggaggtggtgatcttgttcgcgcaggtgggcgaccgcagcatcatgcaaagccagagcctgatgctggagctgcgagagcaggaccaggtgtgggtacgcctctacaagggcgaacgtgagaacgccatcttcagcgaggagctggacacctacatcaccttcagtggctacctggtcaagcacgccaccgagccctagctggccggccacctcctttcctctcgccaccttccacccctgcgctgtgctgaccccaccgcctcttccccgatccctggactccgactccctggctttggcattcagtgagacgccctgcacacacagaaagccaaagcgatcggtgctcccagatcccgcagcctctggagagagctgacggcagatgaaatcaccagggcggggcacccgcgagaaccctctgggaccttccgcggccctctctgcacacatcctcaagtgaccccgcacggcgagacgcgggtggcggcagggcgtcccagggtgcggcaccgcggctccagtccttggaaataattaggcaaattctaaaggtctcaaaaggagcaaagtaaaccgtggaggacaaagaaaagggttgttatttttgtctttccagccagcctgctggctcccaagagagaggccttttcagttgagactctgcttaagagaagatccaaagttaaagctctggggtcaggggaggggccgggggcaggaaactacctctggcttaattcttttaagccacgtaggaactttcttgagggataggtggaccctgacatccctgtggccttgcccaagggctctgctggtctttctgagtcacagctgcgaggtgatgggggctggggccccaggcgtcagctcccagagggacagctgagccccctgccttggctccaggttggtagaagcagccgaagggctcctgacagtggccagggacccctgggtcccccaggcctgcagatgtttctatgaggggcagagctcctggtacatccatgtgtggctctgctccacccctgtgccaccccagagccctggggggtggtctccatgcctgccaccctggcatcggctttctgtgccgcctcccacacaaatcagccccagaaggccccggggccttggcttctgttttttataaaacacctcaagcagcactgcagtctcccatctcctcgtgggctaagcatcaccgcttccacgtgtgttgtgttggttggcagcaaggctgatccagaccccttctgcccccactgcgctcatccaggcctctgaccagtagcctgagaggggctttttctaggcttcagagcangggagagctggacggggtagacagtccgcttgtctgttctaagctctgtgagctcagtctgagacaa

SEQ ID NO: 2 - DNA, 2481 bp, Homo sapiens
  627722CB1
ctagcaagcaggtaaacgagctttgtacaaacacacacagaccaacacatccggggatggctgtgtgttgctagagcagaggctgattaaacactcagtgtgttggctctctgtgccactcctggaaaataatgaattgggtaaggaacagttaataagaaaatgtgccttgctaactgtgcacattacaacaaagagctggcagctcctgaaggaaaagggcttgtgccgctgccgttcaaacttgtcagtcaactcatgccagcagcctcagcgtctgcctccccagcacaccctcattacatgtgtctgtctggcctgatctgtgcatctgctcggagacgctcctgacaagtcgggaatttctctatttctccactggtgcaaagagcggatttctccctgcttctcttctgtcacccccgctcctctcccccaggaggctccttgatttatggtagctttggacttgcttccccgtctgactgtccttgacttctagaatggaagaagctgagctggtgaagggaagactccaggccatcacagataaaagaaaaatacaggaagaaatctcacagaagcgtctgaaaatagaggaagacaaactaaagcaccagcatttgaagaaaaaggccttgagggagaaatggcttctagatggaatcagcagcggaaaagaacaggaagagatgaagaagcaaaatcaacaagaccagcaccagatccaggttctagaacaaagtatcctcaggcttgagaaagagatccaagatcttgaaaaagctgaactgcaaatctcaacgaaggaagaggccattttaaagaaactaaagtcaattgagcggacaacagaagacattataagatctgtgaaagtggaaagagaagaaagagcagaagagtcaattgaggacatctatgctaatatccctgaccttccaaagtcctacataccttctaggttaaggaaggagataaatgaagaaaaagaagatgatgaacaaaataggaaagctttatatgccatggaaattaaagttgaaaaagacttgaagactggagaaagtacagttctgtcttcaatacctctgccatcagatgactttaaaggtacaggaataaaagtttatgatgatgggcaaaagtcagtgtatgcagtaagttctaatcacagtgcagcatacaatggcaccgatggcctggcaccagttgaagtagaggaacttctaagacaagcctcagagagaaactctaaatccccaacagagtatcatgagcctgtatatgccaatcccttttacaggcctacaaccccacagagagaaacggtgacccctggaccaaactttcaagaaaggataaagattaaaactaatggactgggtattggtgtaaatgaatccatacacaatatgggcaatggtctttcagaggaaaggggaaacaacttcaatcacatcagtcccattccgccagtgcctcatccccgatcagtgattcaacaagcagaagagaagcttcacaccccgcaaaaaaggctaatgactccttgggaagaatcgaatgtcatgcaggacaaagatgcaccctctccaaagccaaggctgagccccagagagacaatatttgggaaatctgaacaccagaattcttcacccacttgtcaggaggacgaggaagatgtcagatataatatcgttcattccctgcctccagacataaatgatacagaaccggtgacaatgattttcatggggtatcagcaggcagaagacagtgaagaagataagaagtttctgacaggatatgatgggatcatccatgctgagctggttgtgattgatgatgaggaggaggaggatgaaggagaagcagagaaaccgtcctaccaccccatagctccccatagtcaggtgtaccagccagccaaaccaacaccacttcctagaaaaagatcagaagctagtcctcatgaaaacacaaatcataaatccccccacaaaaattccatatctctgaaagagcaagaagaaagcttaggcagccctgtccaccattccccatttgatgctcagacaactggagatgggactgaggatccatccttaacagctttaaggatgagaatggcaaagctgggaaaaaaggtgatctaagagttgtaccacctatataaacatcctttgaagaagaaactaagaagcatttgcaaatttctcttctggatattttgtttattttttctgaagtccaaaaaattatcattacagtgtaccatattaagccatgtgaataagtagtagtcattatttgtgaaaaattcccaaaaagctggggaaaacaaatgtgtaacttttccagttacttgacacgattcagtgggggaaaaccagcattttttattctattgataccaaagcatttctaataagagcttgttaaatttaagaataaagttatttaaaataaaaaaaaaaa

SEQ ID NO: 3 - DNA, 2987 bp, Homo sapiens
  unsure 2955: a or g or c or t, unknown, or other
agaaaaaaagaaaaaagaaaaaaactaaggcagcagctcttaataaataacacctggagcagaatcggtaaactgctttcacgttggcttttgcagaagtggcaatgcattgaggatacatctggcaagcttcgaattcacaagtgtaaaggacccagtgacctgctcacagtccggcagagcacgcggaacctctacgctcgcggcttccatgacaaagacaaagagtgcagttgtagggagtctggttaccgtgccagcagaagccaaagaaagagtcaacggcaattcttgagaaaccaggggactccaaagtacaagcccagatttgtccatactcggcagacacgttccttgtccgtcgaatttgaaggtgaaatatatgacataaatctggaagaagaagaagaattgcaagtgttgcaaccaagaaacattgctaagcgtcatgatgaaggccacaaggggccaagagatctccaggcttccagtggtggcaacaggggcaggatgctggcagatagcagcaacgccgtgggcccacctaccactgtccgagtgacacacaagtgttttattcttcccaatgactctatccattgtgagagagaactgtaccaatcggccagagcgtggaaggaccataaggcatacattgacaaagagattgaagctctgcaagataaaattaagaatttaagagaagtgagaggacatctgaagagaaggaagcctgaggaatgtagctgcagtaaacaaagctattacaataaagagaaaggtgtaaaaaagcaagagaaattaaagagccatcttcacccattcaaggaggctgctcaggaagtagatagcaaactgcaacttttcaaggagaacaaccgtaggaggaagaaggagaggaaggagaagagacggcagaggaagggggaagagtgcagcctgcctggcctcacttgcttcacgcatgacaacaaccactggcagacagccccgttctggaacctgggatctttctgtgcttgcacgagttctaacaataacacctactggtgtttgcgtacagttaatgagacgcataattttcttttctgtgagtttgctactggctttttggagtattttgatatgaatacagatccttatcagctcacaaatacagtgcacacggtagaacgaggcattttgaatcagctacacgtacaactaatggagctcagaagctgtcaaggatataagcagtgcaacccaagacctaagaatcttgatgttggaaataaagatggaggaagctatgacctacacagaggacagttatgggatggatgggaaggttaatcagccccgtctcactgcagacatcaactggcaaggcctagaggagctacacagtgtgaatgaaaacatctatgagtacagacaaaactacagacttagtctggtggactggactaattacttgaaggatttagatagagtatttgcactgctgaagagtcactatgagcaaaataaaacaaataagactcaaactgctcaaagtgacgggttcttggttgtctctgctgagcacgctgtgtcaatggagatggcctctgctgactcagatgaagacccaaggcataaggttgggaaaacacctcatttgaccttgccagctgaccttcaaaccctgcatttgaaccgaccaacattaagtccagagagtaaacttgaatggaataacgacattccagaagttaatcatttgaattctgaacactggagaaaaaccgaaaaatggacggggcatgaagagactaatcatctggaaaccgatttcagtggcgatggcatgacagagctagagctcgggcccagccccaggctgcagcccattcgcaggcacccgaaagaacttccccagtatggtggtcctggaaaggacatttttgaagatcaactatatcttcctgtgcattccgatggaatttcagttcatcagatgttcaccatggccaccgcagaacaccgaagtaattccagcatagcggggaagatgttgaccaaggtggagaagaatcacgaaaaggagaagtcacagcacctagaaggcagcgcctcctcttcactctcctctgattagatgaaactgttaccttaccctaaacacagtatttctttttaacttttttatttgtaaactaataaaggtaatcacagccaccaacattccaagctaccctgggtacctttgtgcagtagaagctagtgagcatgtgagcaagcggtgtgcacacggagactcatcgttataatttactatctgccaagagtagaaagaaaggctggggatatttgggttggcttggttttgattttttgcttgtttgtttgttttgtactaaaacagtattatcttttgaatatcgtagggacataagtatatacatgttatccaatcaagatggctagaatggtgcctttctgagtgtctaaaacttgacacccctggtaaatctttcaacacacttccactgcctgcgtaatgaagttttgattcatttttaaccactggaatttttcaatgccgtcattttcagttagatgattttgcactttgagattaaaatgccatgtctatttgattagtcttatttttttatttttacaggcttatcagtctcactgttggctgtcattgtgacaaagtcaaataaacccccaaggacgacacacagtatggatcacatattgtttgacattaagcttttgccagaaaatgttgcatgtgttttacctcgacttgctaaaatcgattagcagaaaggcatggctaataatgttggtggtgaaaataaataaataagtaaacaaaaaaaaaaaaaaaaaaaaaaaaaaaaaaaaaaaaaaaaaaaaaaaaaaaaaagcaaaaaaagctgccgccacagttagatgaagaagcatgaggatccgagngggtcgcctctttgagtggtgagggagtcgcg

SEQ ID NO: 4 - DNA, 2915 bp, Homo sapiens
  1362659CB1
gaggcaagaattcggcacgagggacattttgccaacttaaacgagaaaaagaccccccgcacccggcacactcccccttcctccagccccgcttcagccacatgctccagctgctgcccagtaaagccctgtgcctttttttcccctgaatactgcccaaagcatccccttcccatctgcctctcaggagttggggactttgctaggagattttttaagtgttccttactgggacaacgtggagccacgtttgcaggagctccatttgtatccctgctggtgttgacttctgtgtaggggccagttcatgtccctgactctcacctcccattagataaatgaagcccacccccctttctagagtgatgagagtcaagaagaggggatgtatgaacggccaaattcccatgtgagaggaagatgacctgatccacctagccttttcttctggatctgtcctccctcacccctttcacctgagctgtccacagtaggaaacataaagaaacaatgtcccctacatatccccatgactacataatccatcatcgtaggaaataggaaagcaaatttgattttggttttgtaaaacgtacatgcttcaataattctttttttgtgtcttaaatactcataggggaaaaaaacagctcacccaaggtgttaggtttcacatatatattcatcaactattttagaagatttaattctatcaaatcttgtattacctcagatcattttaaatagcaagccaataacgagctttgaaggctattttaccattcctgttcacaaaaggttctcatggtgcctgacaggttacccttgagggcttgtgtctactttttaaaagtcaatggttttttttcttgtgttctagtttccataataggagagaaaatatagaaatatatgcaaaaattatagttttctttagatcagaaactgatatttttgggtcagccatatgtattttgtttaaaggatttaaaataaagtgccgtcatgtagccctgtggaagggagcacataaccagctgtttggcatgacaggtgacttagtatatttgtaattggttttaaaaccaatacaccatactttctttctgcaaacagccatctttatacttagggaagaaaaattgttgggttctagacttttttaatataaattttgttgatatggaattaggtaagtttaagtgtctatgtgcatatgttttttatataagttttttctattcagtttcactgatccaactggcagtgggtaaatatggcataagttaataacacttttccccaaaatggtgctttggatttgaaaagggtctgatggggagaaggagaacgtatcatcctagcttcctctcttaataaacctagaaaaacgggtagtaaactgtggatagtcaggaaaacacccagcaagggacacagctgtcaggaaatgaatcttccccccaacccccaccatgcagatggatagacagaatctttcctgactagtcattaggatcaggggcctctgttggatttgtgtttcttgaagaatagctggcagagtggtataaaagacacgaatatctcctggtctataaggatactctgatttggggtttgcatttttcatggtttttatttcctgttccccctggagttttccattagtgagtttttgtgcaaggatcttatttgtgatgccttccctcccctagaaagattttgtgcaatatattaaatggggacagaattctaaatggataaaacaatggctggttctagccctgagtgacagtcttaaggctagatccttcccatagtatcatctgtcctctggaatgactctcctgtccctaaaggggttaagagagagatcacctagaaatccctctggacacttgtgggttctttagggtttgagtttcttcttccccttgagcttcagagaggagagttggcatggttaaatctgaatggttacctcactgctgaaaacccagaggggcgtggcacactcgcttgtgtggaaaagcctctaaatgcatcccttcctttctttcctgcttcctttgccttacaattgaagcagcccgtggtaccatcacagtatgcagagacttcctcacctttcatatctagggaccacccccgatgcattggtgagggtgggcacttataaatgcctgctattgttaagccattccagcctcttcctctgaatagaccagacgccctttcacttagttcagtgccagtccttttgccttcccaaccctgctgttaggcctgctgttccctttgctcttgattaggagagatggaaggagatgagctcccataactgaattggcctttggttcatgttttctccccatatgtatatatgccatatgtgaatatgccatatatatgtgccaacaaatctatctacgttgttcttttcaaattagcacgcagataggaattttgagtttcttcttcttttagtaactagtataacaagcactggtatttttgtacaaaaaagaaaaacaaaagattgactattgtggtctgcatgacataaacaaacaaatggtgatatcaaagcaacgtataccccagtccagtgtgtgttgccataatttgcaattcagcttaacagtgcacccaatctatatttgcattttgatattatttaagctctatgtacaaggttttgcatgtatttatatggttcttagggaaaaaaaatgctataaactgcaaatctgaaattcaaatgtgttgttccactgagaccagaagaagaagaggagttttaaaagggataatttgttggagccaataaagctttttgctgatgaacagaaaccaatactgctgtgcactgagaataaaaactcatgcccacttgtaaaaaaaaaaaaagg

SEQ ID NO: 5 - DNA, 1826 bp, Homo sapiens
  1446685CB1
gaaagccgcagcctcagtcccgccgccgcccgctgcgtccgcccagcgccagctccgcgtcccgaccggcccgcggcagcctgcgccgcgccatggccacctccccgcagaagtcgccttctgtccccaagtctcccactcccaagtcgcccccgtcccgcaagaaagatgattccttcttggggaaactcggagggaccctggcccggaggaagaaagccaaggaggtgtccgagctgcaggaggagggaatgaacgccatcaacctgcccctcagcccaattccctttgagctggaccccgaggacacgatgctggaggagaatgaggtgcgaacaatggtggatccaaactcacgcagtgaccccaagcttcaagaactgatgaaggtattaattgactggattaatgatgtgttggttggagaaagaatcattgtgaaagacctagctgaagatttgtatgatggacaagtcctgcagaagcttttcgagaaactggagagtgagaagctaaatgtggctgaggtcacccagtcagagattgctcagaagcaaaaactgcagactgtcctggagaagatcaatgaaaccctgaaacttcctcccaggagcatcaagtggaatgtggattctgttcatgccaagagcctggtggccatcttacacctgctcgttgctctgtctcagtatttccgcgcaccaattcgactcccagaccatgtttccatccaagtggttgtggtccagaaacgagaaggaatcctccagtctcggcaaatccaagaggaaataactggtaacacagaggctctttccgggaggcatgaacgtgatgcctttgacaccttgttcgaccatgccccagacaagctgaatgtggtgaaaaagacactcatcactttcgtgaacaagcacctgaataaactgaacctggaggtcacagaactggaaacccagtttgcagatggggtgtacctggtgctgctcatggggctcctggagggctactttgtgcccctgcacagcttcttcctgaccccggacagctttgaacagaaggtcttgaatgtctcctttgcctttgagctcatgcaagatggagggttggaaaagccaaaaccgcggccagaagacatagtcaactgtgacctgaaatctacactacgagtgttgtacaacctcttcaccaagtaccgtaacgtggagtgaggggctgccctgggcccaccactgcccaagagttcttgctgttggcgtactggaccctcctccgaactgccttaccctgcttattcctgtctcttgcactgtgctctcccacaagtccagctgcaacccagagatagtggaaactgaaattaggaaggaaatcatcaataactcagtgggctgacccatccctcccaggcgctggggaccaacctagcaatgaaggttgggaaggttgttcccttcccggtgccaggtccagatttccctccatgatttgggaaccaggttaggcaaaagagtccccacaagatgaaaataaagatcctagttaccattcaaaggatgctaactgtgtgtcaggccccacactaagtgctctgctctgatatactcaaggccattaatcttcaggactcccattgacgtaggtgtttcattccccttttacagatgaggaaactaaggcttggaggttaaatgacttgccagaagttggaatttttttcctctttgaacataacctctcccttctccctaaaggtaaccactattctgagtccaatcatcaaggttttgcttttctttttagctaagtatgcattcctcaatagt

SEQ ID NO: 6 - DNA, 1439 bp, Homo sapiens
  1556751CB1
gagtatcccttgtttaatcacttttgtggttaaaagagacctttgggtcagtctgcctcattccttgaagagtttagccctggctcacttttcactctatttcttctcctgtctcaagaaagaagaaaaaaagagacaaattacccagaaacccctcccttccccacatggaggccttggcaaatgttaattttcctagaaaatccttcagacctgaagacgcaggaaaagaatctggctctcagggtggcttctgcgtccccgccgccaggccccagactatggtcacagggccgtcctgttcctccccgggactccagaatttctctcctcaaaggaaagaaaacagggcatgcgcttgttggcaaaacgcagggccggctcccaaaaaccccatgtgtgtacgattaaaagttggccgtccccaggcctcccagcgcaaacttaaagagacagggctttgctgaaaaccaaacatgggccagctgggctttttaacaacctagagactttccggagctgcctggaacagagcctgcgggaaacggggcttgccagagacactcacagtttccttcatggcctgttttggtcccctaagaatctccacatcattgtctttcttgtgccttttccttggtgagcaacagaaagggaagggttccaagcctctaaaaatgtgctttgtgatcaggagtgcgctccaaaccaaatacgcgcgctgccctttcgaggccagtgagctcagcctccaaggctttaaagccacatttcagcaagagaaagcgctgagagctcgcaggttcattaaagaaggcaaagcactggtttctctccttagaaaagtaggtttcttggcttgatgtagactggcttgctttgatttttagtgaagggaatgtacgtaaaacaaaatagggcttggctggtcaaaggagacaagcaggatggatggatggatggatggatggatgtatggatgaatagatagatggtgtttgcatgtaaattgcagagaaaacaaaaccaaagctgattggaaacaattaattgtgggtgtctgagggggaaggtcgcagctttgggcagctttgagaagcggtacaagagttctgtgcctgtgtgtccagccctggagccagccagtgcatttattttaagctcttagaagcaactccttggcccaggaatgcgtgacccctgagatgggtccacgcatctctctacacttccttctctccgtgggatactggactcgtgcctctgcgcccattctcttctcacgcatatccatgagctttaatttcactttctgatcacggtacgtccataaagccagtattacacttaaatgaagtattcttttttgtaatcgttttttttagaaggtaaacaaatttaataaagctaccaataatgagaaaaaaaaaaaaaaaaaa

SEQ ID NO: 7 - DNA, 3047 bp, Homo sapiens
  1656953CB1
cgagacagaggaaatgtgtctccctccaaggccccaaagcctcagagaaagggtgtttctggttttgccttagcaatgcatcggtctctgaggtgacactctggagcggttgaagggccacaaggtgcagggttaatactcttgccagttttgaaatatagatgctatggttcagattgtttttaatagaaaactaaaggggcaggggaagtgaaaggaaagatggaggttttgtgcggctcgatggggcatttggaacttctttttaaagtcatctcatggtctccagttttcagttggaactctggtgtttaacacttaagggagacaaaggctgtgtccatttggcaaaacttccttggccacgagactctaggtgatgtgtgaagctgggcagtctgtggtgtggagagcagccatctgtctggccattcagaggattctaaagacatggctggatgcgctgctgaccaacatcagcacttaaataaatgcaaatgcaacatttctccctctgggccttgaaaatccttgcccttatcatttggggtgaaggagacatttctgtccttggcttcccacagccccaacgcagtctgtgtatgattcctgggatccaacgagccctcctattttcacagtgttctgattgctctcacagcccaggcccatcgtctgttctctgaatgcagccctgttctcaacaacagggaggtcatggaacccctctgtggaacccacaaggggagaaatgggtgataaagaatccagttcctcaaaaccttccctggcaggctgggtccctctcctgctgggtggtgctttctcttgcacaccactcccaccacggggggagagccagcaacccaaccagacagctcaggttgtgcatctgatggaaaccactgggctcaaacacgtgctttattctcctgtttatttttgctgttactttgaagcatggaaattcttgtttgggggatcttggggctacagtagtgggtaaacaaatgcccaccggccaagaggccattaacaaatcgtccttgtcctgaggggccccagcttgctcgggcgtggcacagtggggaatccaagggtcacagtatggggagaggtgcaccctgccacctgctaacttctcgctagacacagtgtttctgcccaggtgacctgttcagcagcagaacaagccagggccatggggacgggggaagttttcacttggagatggacaccaagacaatgaagatttgttgtccaaataggtcaataattctgggagactcttggaaaaaactgaatatattcaggaccaactctctccctcccctcatcccacatctcaaagcagacaatgtaaagagagaacatctcacacacccagctcgccatgcctactcattcctgaatttcaggtgccatcactgctctttctttcttctttgtcatttgagaaaggatgcaggaggacaattcccacagataatctgaggaatgcagaaaaaccagggcaggacagttatcgacaatgcattagaacttggtgagcatcctctgtagagggactccacccctgctcaacagcttggcttccaggcaagaccaaccacatctggtctctgccttcggtggcccacacacctaagcgtcatcgtcattgccatagcatcatgatgcaacacatctacgtgtagcactacgacgttatgtttgggtaatgtggggatgaactgcatgaggctctgattaaggatgtggggaagtgggctgcggtcactgtcggccttgcaaggccacctggaggcctgtctgttagccagtggtggaggagcaaggcttcaggaagggccagccacatgccatcttccctgcgatcaggcaaaaaagtggaattaaaaagtcaaacctttatatgcatgtgttatgtccattttgcaggatgaactgagtttaaaagaatttttttttctcttcaagttgctttgtcttttccatcctcatcacaagcccttgtttgagtgtcttatccctgagcaatctttcgatggatggagatgatcattaggtacttttgtttcaacctttattcctgtaaatatttctgtgaaaactaggagaacagagatgagatttgacaaaaaaaaattgaattaaaaataacacagtctttttaaaactaacataggaaagcctttcctattatttctcttcttagcttctccattgtctaaatcaggaaaacaggaaaacacagctttctagcagctgcaaaatggtttaatgccccctacatatttccatcaccttgaacaatagctttagcttgggaatctgagatatgatcccagaaaacatctgtctctacttcggctgcaaaacccatggtttaaatctatatggtttgtgcattttctcaactaaaaatagagatgataatccgaattctccatatattcactaatcaaagacactattttcatactagattcctgagacaaatactcactgaagggcttgtttaaaaataaattgtgttttggtctgttcttgtagataatgcccttctattttaggtagaagctctggaatccctttattgtgctgttgctcttatctgcaaggtggcaagcagttcttttcagcagattttgcccactattcctctgagctgaagttctttgcatagatttggcttaagcttgaattagatccctgcaaaggcttgctctgtgatgtcagatgtaattgtaaatgtcagtaatcacttcatgaacgctaaatgagaatgtaagtatttttaaatgtgtgtatttcaaatttgtttgactaattctggaattacaagatttctatgcaggatttaccttcatcctgtgcatgtttcccaaactgtgaggagggaaggctcagagatcgagcttctcctctgagttctaacaaaatggtgctttgagggtcagcctttaggaaggtgcagctttgttgtcctttgagctttctgttatgtgcctatcctaataaactcttaaacacaaaaaaaaaaa

SEQ ID NO: 8 - DNA, 3017 bp, Homo sapiens
  1662318CB1
cgcaaactcaaccctttcggaaacaccttcctcaacaggttcatgtgtgcccagctccctaatcaggtcctggagagcatcagcatcatcgacaccccgggtatcctgtcgggtgccaagcagagagtgagccgcggctacgacttcccggccgtgctgcgctggttcgcggagcgcgtggacctcatcatcctgctctttgatgcgcacaagctggagatctcggacgagttctcagaggccatcggcgcgttgcggggccatgaggacaagatccgcgtggtgctcaacaaggccgacatggtggagacgcagcagctgatgcgcgtctacggcgcgctcatgtgggcgctgggcaaggtggtgggcacgcccgaggtgctgcgcgtctacatcggctccttctggtcccagcccctcctggtgcccgacaaccggcgcctcttcgagctggaggagcaggacctcttccgcgacatccagggcctgccccggcacgcagccttgcgcaagctcaacgacctggtgaagagggcccggctggtgcgagttcacgcttacatcatcagctacctgaagaaggagatgccctctgtgtttgggaaggagaacaagaagaagcagctgatcctcaaactgcccgtcatctttgcgaagattcagctggaacatcacatctcccctggggactttcctgattgccagaaaatgcaggagctgctgatggcgcacgacttcaccaagtttcactcgctgaagccgaagctgctggaggcactggacgagatgctgacgcacgacatcgccaagctcatgcccctgctgcggcaggaggagctggagagcaccgaggtgggcgtgcaggggggcgcttttgagggcacccacatgggcccgtttgtggagcggggacctgacgaggccatggaggacggcgaggagggctcggacgacgaggccgagtgggtggtgaccaaggacaagtccaaatacgacgagatcttctacaacctggcgcctgccgacggcaagctgagcggctccaaggccaagacctggatggtggggaccaagctccccaactcagtgctggggcgcatctggaagctcagcgatgtggaccgcgacggcatgctggatgatgaagagttcgcgctggccagccacctcatcgaggccaagctggaaggccacgggctgcccgccaacctgccccgtcgcctggtgccaccctccaagcgacgccacaagggctccgccgagtgagccgggcccccctcccatggccctgctgtggctccccagctccagtcggctgcacgcacacccctgctccggctcacacacgccctgcctgccctccctgcccagctgtaaggaccgggggtctccctcctcactaccgccagacaccccggtggaagcatttagaggggaccacgggagggacaaggcttctctgtccgcccttcacacctccagcctcacgttcacttaggcacatcacacacacactggcacacgcaggcatccatccatccgtcattcattcaaatatttattgagcacctactatgtgcccagccctgttctaggcactgggcattaccatagagaacaaaatagacaaatacatctgccctcatggaaggtgacgttcccaggagagggcacctacacagtcacgcaaacacacactaattcctggcagggcccccagcccctcccctggctgagcagccctgtggctgaaatgactagcagataaacagacccccttctgctccgcttcctcctgcccagccaggcaacaccctcaaccggctccatcacatcctcaggtctcgggaccatggggggctcagaggggagacacacctactgcttcctcagatgggcccctccgcagccccttcccttgctcggggaaagcccccaattctgcccacacccatttatttccttccttccttccttcttttctttccttccttccttcttttttgtttttgcccccaattctgcccatacccatttctttctttccttccttccttcttttttgtttttgcccccagttctgtccacaccccttccctttcctgtcctgtcctttctttcttttttgatagaatcttgctctgtcgcccaggctgggagtgcagtggtgagatctcagctcactgcaacctccacctcctgggttgaagtgattctcgtgcctcagcctcctgagtagctgggactgcaggcacgcgccaccacgcccagctaatttttgtatttgagtagagacggggtttcaccatgttggccaggctggtctcgaactccgcatctcaggtgatctgctcgcctcggcctcccaaagtgatgggattacaggcatgagccaccgtgcccggcttcacacccatttctttaaaaaggatcccgtagcaggcagaaaagccccttccatcctgctcctctgatactgtgcccccttggagatatttccgtcctccacccacgtgtctgtggctggaactgcccagcctgctcctggccccctggaagcctccccacagctggtaatctggacttaaggattgctgggccaccgcctctctgcctaccaccattccatatttaagtggagcccctacgtagaaaggccccggggctttattttagtctccttttcagggatgtcgtgggcgggggagggggttcttggtgctacagccctctccccacccctaaagggacgccgacgctgtttgctgccttcaccacatattagtgcttgaccctggcaggggaccccatggaaaagatggggaagagcaaaatacatggagacgacgcaccctccaggatgctcgctgggattcccacgcccaccactgtcccccaccccatggctgggaggggcctctgaacggaacagtgtccccacagagcgaataaagcaaggcttcttccccaaaaaaaaaaaaaaaaaaaaaattggtgcggccgaagttattcccttc

SEQ ID NO: 9 - DNA, 1735 bp, Homo sapiens
  1996726CB1
tcgggaggaaggagactacacctgctttgctgaaaatcaggtcgggaaggacgagatgagagtcagagtcaaggtggtgacagcgcccgccaccatccggaacaagacttacttggcggttcaggtgccctatggagacgtggtcactgtagcctgtgaggccaaaggagaacccatgcccaaggtgacttggttgtccccaaccaacaaggtgatccccacctcctctgagaagtatcagatataccaagatggcactctccttattcagaaagcccagcgttctgacagcggcaactacacctgcttggtcaggaacagcgcgggagaggataggaagacggtgtggattcacgtcaacgtccagccacccaagatcaacggtaaccccaaccccatcaccaccgtgcgggagatagcagccgggggcagtcggaaactgattgactgcaaagctgaaggcatccccaccccgagggtgttatgggcttttcccgagggtgtggttctgccagctccatactatggaaaccggatcactgtccatggcaacggttccctggacatcaggagtttgaggaagagcgactccgtccagctggtatgcatggcacgcaacgagggaggggaggccaggttgatcgtgcagctcactgtcctggagcccatggagaaacccatcttccacgacccgatcagcgagaagatcacggccatggcgggccacaccatcagcctcaactgctctgccgcggggaccccgacacccagcctggtgtgggtccttcccaatggcaccgatctgcagagtggacagcagctgcagcgcttctaccacaaggctgacggcatgctacacattagcggtctctcctcggtggacgccggggcctaccgctgcgtggcccgcaatgccgctggccacacggagaggctggtctccctgaaggtgggactgaagccagaagcaaacaagcagtatcataacctggtcagcatcatcaatggtgagaccctgaagctcccctgcacccctcccggggctgggcagggacgtttctcctggacgctccccaatggcatgcatctggagggcccccaaaccctgggacgcgtttctcttctggacaatggcaccctcacggttcgtgaggcctcggtgtttgacaggggtacctatgtatgcaggatggagacggaatacggcccttcggtcaccagcatccccgtgattgtgatcgcctatcctccccggatcaccagcgagcccaccccggtcatctacacccggcccgggaacaccgtgaaactgaactgcatggctatggggattcccaaagctgacatcacgtgggagttaccggataagtcgcatctgaaggcaggggttcaggctcgtctgtatggaaacagatttcttcacccccagggatcactgaccatccagcatgccacacagagagatgccggcttctacaagtgcatggcaaaaaacattctcggcagtgactccaaaacaacttacatccacgtcttctgaaatgtggattccagaatgattgcttaggaactgacaacaaagcggggtttgtaagggaagccaggttggggaataggagctcttaaataatgtgtcacagtgcatggtggcctctggtgggtttcaagttgaggttgatcttgatctacaattgttgggaaaaggaagcaatgcagacacgagaaggagggctca

SEQ ID NO: 10 - DNA, 1016 bp, Homo sapiens
  2137155CB1
ctgtacgttcccctgtggcccacgcctagtgaaaatgatatcgtacatctccctagagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaagccttcttccttaaataatcagctagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttgcttagtggattgtattggattgtgacttgatgtacagcgctaagaccttactgggatgggctctgtctacagcaatgtgcagaacaagcattcccacttttcctcaagataactgaccaagtgtttcttagaaccaaagtttttaaagttgctaagatatatttgcctgtaagatagctgtagagatatttggggtggggacagtgagtttggatggcgaaatacaccgcacggtggtgttgggaagaaaaatttgtcagcttggctcggggagaaaccctggtacactaaagcagttcagtgtgccagaggttatttttttcccattgctctgaagactgcactggttgctgcaaactcaggcctgaatgagcggaaacaaaaaaagccttgcgccccgatgccataacacctttggaatcccgagcggccctcagaaaccttttcaggcatccaggtcttaagcccaagtatctttctatacagtcccactgcggtgagcgtgggggag

SEQ ID NO: 11 - DNA, 2288 bp, Homo sapiens
  2268890CB1
caaccagggtcaggctgtgctcacagtttcctctggcggcatgtaaaggctccacaaaggagttgggagttcaaatgaggctgctgcggacggcctgaggatggaccccaagccctggacctgccgagcgtggcactgaggcagcggctgacgctactgtgagggaaagaaggttgtgagcagccccgcaggacccctggccagccctggccccagcctctgccggagccctctgtggaggcagagccagtggagcccagtgaggcagggctgcttggcagccaccggcctgcaactcaggaacccctccagaggccatggacaggctgccccgctgacggccagggtgaagcatgtgaggagccgccccggagccaagcaggagggaagaggctttcatagattctattcacaaagaataaccaccattttgcaaggaccatgaggccactgtgcgtgacatgctggtggctcggactgctggctgccatgggagctgttgcaggccaggaggacggttttgagggcactgaggagggctcgccaagagagttcatttacctaaacaggtacaagcgggcgggcgagtcccaggacaagtgcacctacaccttcattgtgccccagcagcgggtcacgggtgccatctgcgtcaactccaaggagcctgaggtgcttctggagaaccgagtgcataagcaggagctagagctgctcaacaatgagctgctcaagcagaagcggcagatcgagacgctgcagcagctggtggaggtggacggcggcattgtgagcgaggtgaagctgctgcgcaaggagagccgcaacatgaactcgcgggtcacgcagctctacatgcagctcctgcacgagatcatccgcaagcgggacaacgcgttggagctctcccagctggagaacaggatcctgaaccagacagccgacatgctgcagctggccagcaagtacaaggacctggagcacaagtaccagcacctggccacactggcccacaaccaatcagagatcatcgcgcagcttgaggagcactgccagagggtgccctcggccaggcccgtcccccagccaccccccgctgccccgccccgggtctaccaaccacccacctacaaccgcatcatcaaccagatctctaccaacgagatccagagtgaccagaacctgaaggtgctgccaccccctctgcccactatgcccactctcaccagcctcccatcttccaccgacaagccgtcgggcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgatggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaacggggtctggtaccgcgggggccattaccggagccgctaccaggacggagtctactgggctgagttccgaggaggctcttactcactcaagaaagtggtgatgatgatccgaccgaaccccaacaccttccactaagccagctccccctcctgacctctcgtggccattgccaggagcccaccctggtcacgctggccacagcacaaagaacaactcctcaccagttcatcctgaggctgggaggaccgggatgctggattctgttttccgaagtcactgcagcggatgatggaactgaatcgatacggtgttttctgtccctcctactttccttcacaccagacagcccctcatgtctccaggacaggacaggactacagacaactctttctttaaataaattaagtctctacaataaaaacacaactgcaaagtaccttcataatatacatgtgtatgagcctcccttgtgcacgtatgtgtatagcacatatatatgg

SEQ ID NO: 12 - DNA, 3304 bp, Homo sapiens
  2305981CB1
ccctcttatggattcccagcaagcatcaggaaccattgtgcaaattgtcatcaataacaaacacaagcatggacaagtgtgtgtttccaatggaaagacctattctcatggcgagtcctggcacccaaacctccgggcatttggcattgtggagtgtgtgctatgtacttgtaatgtcaccaagcaagagtgtaagaaaatccactgccccaatcgatacccctgcaagtatcctcaaaaaatagacggaaagtgctgcaaggtgtgtccaggtaaaaaagcaaaagaagaacttccaggccaaagctttgacaataaaggctacttctgcggggaagaaacgatgcctgtgtatgagtctgtattcatggaggatggggagacaaccagaaaaatagcactggagactgagagaccacctcaggtagaggtccacgtttggactattcgaaagggcattctccagcacttccatattgagaagatctccaagaggatgtttgaggagcttcctcacttcaagctggtgaccagaacaaccctgagccagtggaagatcttcaccgaaggagaagctcagatcagccagatgtgttcaagtcgtgtatgcagaacagagcttgaagatttagtcaaggttttgtacctggagagatctgaaaagggccactgttaggcaagacagacagtattggatagggtaaagcaagaaaactcaagctgcagctggactgcaggcttattttgcttaagtcaacagtgccctaaaactccaaactcaaatgcagtcaattattcacgccatgcacagcataatttgctcctttgtgtggagtggtgtgtcagcccttgaacatctcctccaaagagactagaagagtcttaaattatatgtgggaggaggagggatagaacatcacaacactgctctagtttcttggagaatcacatttctttacaggttaaagacaaacaagaccccagggtttttatctagaaagttattcaagtgaaagaaagagaagggaattgcttagtaggagttctgcagtatagaacaattacttgtatgaaattatacctttgaattttagaatgtcatgtgttcttttaaaaaaattagctccccatcctccctcctcactccctccctccctccttctctctctctctctctctccctctctcacagacacacacacacacacacacacacgcacacgcacgtccacactcacattaaactaaagctttatttgaagcaaagctagccaaaattctacgttacttttcccttgactggatcccaagtagcttggaagtttttgtgcccaggagagtaaataactgtgaacaagaggctctgcccttaggtctttgtggctgtttaagtcaccaacaatagagtcagggtaaagaataaaaacactttcatagcctcattcattcacttagaagtggtaataatttttccctaatgataccacttttcttttccccctgtacctatgggacttccagaaagaagttaaattgagtaaaatcatcagaaactgaatccatgtaagaaaaaataattgttgaagaaagaagttgatagaattcaaaaaggccatctttttgctttcacatcaataaaatttaccaagtaatagatcagtactcactaatatttttgagaccatagttgtctggtcagaaaaattatattaaattagtaaattctagaagctctttaaaagggaagttttccttcttctccaattataggagttgatttttactttgcaaagtggctcggtcctcatgagcatctgcatgttgactcttcagttaagaaaattgttgttcatttagggaggtggatattctgatgaagatctttatcctaaaccttcctactatccttgtcttattcatcaagcagatattttagtcaagaattccagagaaggctgctcctaaaatgtctacttgcagcccaataccagagcataaactatccattctggggtctggctttagaaatcatctttgtgggaagacctaattcttcacagcaaggatctcaggcatgccttctagatttgttccctctgaggggcaggaatgaactgtagaaatgttttaaggacccagaaaccccatatgtctcattccatgactataggtgagagaattctttcctaagagggtttgataccaataggggaaaatgtaaaatgttcagtctttatgacaacctggcataaaggagtcaattcttatgaaagagacacaagggccttatggccagggtttcttgggacaagactctcaccagcacatcacacacgttctccttggaagagagaagcagtacatcccggttgagaggtcacaaagcattagtttgtgtgtgtgtgtgtgtgtgtgtgtgtgtgtgtgtgtgtgtgtgtgtgtggtaaaggggggaaggtgttatgcggctgctccctccgtcccagaggtggcagtgattccataatgtggagactagtaactagatcctaaggcaaagaggtgtttctccttctggatgattcatcccaaagccttcccacccaggtgttctctgaaagcttagccttaagagaacacgcagagagtttccctagatatactcctgcctccaggtgctgggacacacctttgcaaaatgctgtgggaagcaggagctggggagctgtgttaagtcaaagtagaaaccctccagtgtttggtgttgtgtagagaataggacatagggtaaagaggccaagctgcctgtagttagtagagaagaatggatgtggttcttcttgtgtatttatttgtatcataaacacttggaacaacaaagaccataagcatcatttagcagttgtagccattttctagttaactcatgtaaacaagtaagagtaacataacagtattaccctttcactgttctcacaggacatgtacctaattatggtacttatttatgtagtcactgtatttctggatttttaaattaataaaaaagttaattttgaaaaaaaaaaaaaaaaaaaaaaaaaaaaaaaaaaaaaaaaaaaactcgagggggggcctgtaccgggttccccgtaacaggttcgcccttaagattccctggccgcagtttttggccgcgttttggggaacctctgggtacccccttagttgctcgctaaaatcccctttcgcagcccgtttaaaggctggggccggccgattgccttcccaatagcctcccatgaatgggaatggaattggaagggaaattttggtaaatccggta

SEQ ID NO: 13 - DNA, 708 bp, Homo sapiens
  2457612CB1
ggaaagccaggaagtgcaggaatcatttcatcagggccaataactacaccacccctgaggtcaacacccaggcctactggaactcccttggagagaatagagacagatgtaaagcaaccaacagttcctgcctctggagaagaactggaaaatataactgactttagctcaagcccaacaagagaaactgatcctcttgggaagccaagattcaaaggacctcatgtgcgatacatccaaaagcctgacaacagtccctgctccattactgactctgtcaaacggttccccaaagaggaggccacagaggggaatgccaccagcccaccacagaacccacccaccaacctcactgtggtcaccgtggaagggtgccccttcatttgtcatcttggactgggaaaagccactaaatgacactgtcactgaatatgaagttatatccagagaaaatgggtcattcagtgggaagaacaagtccattcaaatgacaaatcagacattttccacagtagaaaatctgaaaccaaacacgagttatgaattccaggtgaaacccaaaaacccgcttggtgaaggcccggtcagcaacacagtggcattcagtactgaatcagcggacccagagtgagtgagcagtttctgcaggagagatgcctctggactgaaggccgctttgttcgactcttgctcaggtgtaagggcaac

SEQ ID NO: 14 - DNA, 2040 bp, Homo sapiens
  2814981CB1
cggccagccgccgcgcgctgcagctctccgggacgcccgtgcgccagctgcagaagggcgcctgcccgttgggtctccaccagctgagcagcccgcgctacaagttcaacttcattgctgacgtggtggagaagatcgcaccagccgtggtccacatagagctcttcctgagacacccgctgtttggccgcaacgtgcccctgtccagcggttctggcttcatcatgtcagaggccggcctgatcatcaccaatgcccacgtggtgtccagcaacagtgctgccccgggcaggcagcagctcaaggtgcagctacagaatggggactcctatgaggccaccatcaaagacatcgacaagaagtcggacattgccaccatcaagatccatcccaagaaaaagctccctgtgttgttgctgggtcactcggccgacctgcggcctggggagtttgtggtggccatcggcagtcccttcgccctacagaacacagtgacaacgggcatcgtcagcactgcccagcgggagggcagggagctgggcctccgggactccgacatggactacatccagacggatgccatcatcaactacgggaactccgggggaccactggtgaacctggatggcgaggtcattggcatcaacacgctcaaggtcacggctggcatctcctttgccatcccctcagaccgcatcacacggttcctcacagagttccaagacaagcagatcaaagactggaagaagcgcttcatcggcatacggatgcggacgatcacaccaagcctggtggatgagctgaaggccagcaacccggacttcccagaggtcagcagtggaatttatgtgcaagaggttgcgccgaattcaccttctcagagaggcggcatccaagatggtgacatcatcgtcaaggtcaacgggcgtcctctagtggactcgagtgagctgcaggaggccgtgctgaccgagtctcctctcctactggaggtgcggcgggggaacgacgacctcctcttcagcatcgcacctgaggtggtcatgtgaggggcgcattcctccagcgccaagcgtcagagcctgcagacaacggagggcagcgcccccccgagatcaggacgaaggaccaccgtcggtcctcagcagggcggcagcctcctcctggctgtccggggcagagcggaggctgggcttggccaggggcccgaatttccgcctggggagtgttggatccacatcccggtgccggggagggaagcccaacatccccttgtacagatgatcctgaaagtcacttccaagttctccggatattcacaaaactgccttccatggaggtcccctcctctcctagcttcccgcctctgcccctgtgaacacccatctgcagtatcccctgctcctgcccctcctactgcaggtctgggctgccaagcttcttcccccctgacaaacgcccacctgacctgaggccccagcttccctctgccctaggacttaccaagctgtagggccagggctgctgcctgccagcctggggtccctggaggacaggtcacatctgatccctttggggtgcgggggtggggtccagcccagagcaggcactgagtgaatgccccctggctgcggagctgagccccgccctgccatgaggttttcctccccaggcaggcaggaggccgcggggagcacgtggaaagttggctgctgcctggggaagcttctcctccccaaggcggccatggggcagcctgcagaggacagtggacgtggagctgcggggtgtgaggactgagccggcttccccttcccacgcagctctgggatgcagcagccgctcgcatggaagtgccgcccagaggcatgcaggctgctgggcaccaccccctcatccagggaacgagtgtgtctcaaggggcatttgtgagctttgctgtaaatggattcccagtgttgcttgtactgtatgtttctctactgtatggaaaataaagtttacaagcacaaaaaaaaaaaaaaaaaaaaaaaaaaaagg

SEQ ID NO: 15 - DNA, 2121 bp, Homo sapiens
  3089150CB1
gtaaaagctggttgtgatcgcatcatagactccaaaaagaagtttgataaatgtggtgtttgcgggggaaatggatctacttgtaaaaaaatatcaggatcagttactagtgcaaaacctggatatcatgatatcatcacaattccaactggagccaccaacatcgaagtgaaacagcggaaccagaggggatccaggaacaatggcagctttcttgccatcaaagctgctgatggcacatatattcttaatggtgactacactttgtccaccttagagcaagacattatgtacaaaggtgttgtcttgaggtacagcggctcctctgcggcattggaaagaattcgcagctttagccctctcaaagagcccttgaccatccaggttcttactgtgggcaatgcccttcgacctaaaattaaatacacctacttcgtaaagaagaagaaggaatctttcaatgctatccccactttttcagcatgggtcattgaagagtggggcgaatgttctaagtcatgtgaattgggttggcagagaagactggtagaatgccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaagaagcttgaagtgtctgtcccatgatggaggggtgttatctcatgagagctgtgatcctttaaagaaacctaaacatttcatagacttttgcacaatggcagaatgcagttaagtggtttaagtggtgttagctttgagggcaaggcaaagtgaggaagggctggtgcagggaaagcaagaaggctggagggatccagcgtatcttgccagtaaccagtgaggtgtatcagtaaggtgggattatgggggtagatagaaaaggagttgaatcatcagagtaaactgccagttgcaaatttgataggatagttagtgaggattattaacctctgagcagtgatatagcataataaagccccgggcattattattattatttcttttgttacatctattacaagtttagaaaaaacaaagcaattgtcaaaaaaagttagaactattacaacccctgtttcctggtacttatcaaatacttagtatcatgggggttgggaaatgaaaagtaggagaaaagtgagattttactaagacctgttttactttacctcactaacaatggggggagaaaggagtacaaataggatctttgaccagcactgtttatggctgctatggtttcagagaatgtttatacattatttctaccgagaattaaaacttcagattgttcaacatgagagaaaggctcagcaacgtgaaataacgcaaatggcttcctctttccttttttggaccatctcagtctttatttgtgtaattcattttgaggaaaaaacaactccatgtatttattcaagtgcattaaagtctacaatggaaaaaaagcagtgaagcattagatgctggtaaaagctagaggagacacaatgagcttagtacctccaacttcctttctttcctaccatgtaaccctgctttgggaatatggatgtaaagaagtaacttgtgtctcatgaaaatcagtacaatcacacaaggaggatgaaacgccggaacaaaaatgaggtgtgtagaacagggtcccacaggtttggggacattgagatcacttgtcttgtggtggggaggctgctgaggggtagcaggtccatctccagcagctggtccaacagtcgtatcctggtgaatgtctgttcagctcttctgtgagaatatgattttttccatatgtatatagtaaaatatgttactataaattacatgtactttataagtattggtttgggtgttccttccaagaaggactatagttagtaataaatgcctataataacatatttatttttatacatttatttctaatgaaaaaaacttttaaattatatcgcttttgtggaagtgcatataaaatagagtatttatacaatatatgttactagaaataaaagaacacttttggaaaaaaaaaaaaaaa

SEQ ID NO: 16 - DNA, 2900 bp, Homo sapiens
  3206667CB1
gaagttttaaaaaaaactacagcagccaaagaaactatatatatatatatatatatccagaatgattgcctctactgtcctcattgacttgtttgaaccttagtgccttaccctgtcctcttcccagttctctttatagaagctctaggagctttcgaaaagccaaagtctttctgaagaatctgtgctggacagacataattccctttctcattgtctccatctttgttggtcatggtaaggtttttccatcagcctctgaaaaaatagttgtgcacaacatctgctcactggactgtctgatccaatgtaattggctgcgtctggctaattctaagcactaaagtctacatctaagctatagatttaagcttgaagctacagattatatcactatcaccaccacccctcacccagtgaaatcagacagtcagtcatcttaagttaaagatatttgttgtctttgaatgatttgctgtcacagactatttggtagaagaaatatttttcacctgagagaggaagagaaatttctctagtaacacaaagagtgagttctaaaaggcatgcccacatctctttcgtgccttaaggatagtgagatgcacacttatatatatactgtatatatttatatatttatatatatatttcatatatatatataatattgcaagcttaagtttgcaatttcccaaacaatacaaaaagcaaattacacaccctcaccactgttcttatctctatagtgatgaaacattaattagggatcttgctgcttttctttttctacacgaagttttcattaaagccacagaataattgatagggcagctgtttgagaacaggtcccattttcacattagggctttaaatgaattagaaactatttgaggctataaaaatgtccttgagtttggagcctgagctctggtgaaatgctgatacatctgatctatcatgggaattgcagttagagagagtaaggaataccatttagtcatctatccgttcttcacttagcaggaatatgaaagaaaggcacatgtttaagaggaatacctaaaggtttttctaaattccaacatttaaaaggcaattgtgggctatttttattttttaatattttgaaataaagtttagtgtctagggctgggagccaggactgatcttccatttctttttctttgttcccagccatgcttttgtaacttgccaggtggacttgaccaactacattaccatgctgtgcctcagtttacccatttgtaaaatgggattaataatacttacctacctcacaggggtgttgtgaggctctattcatttgctcctttattctttcctgtattctctgtatgtccagcactttgtagccatgggaggaaagggactataaaagtgtacaatgttaatggaatgatacggtacctgaaagccttgttttctagtaagaaaatgctaccttgctgtacatacttataaccttgtatttggaaatgagaaataggtttatattttcagatctctcaaaaatcacatcatttgaccaaagaataatttaagacacatagaacagatttttttaatttatattttcatcctgaccagcttagttctaataatttttagttgtgagtgattaaaaaactttggatcaattttggtcaaacatgccaactttgtagtctgagtgacaggcaaggatttttgggtttaagatgcacttttagcacacatttgtatttcccttggcatatcagattgagctaatggtgatgttatttcaatctaacagccaccaatctgaaattgtatttcaaatgttgattctgtagttctttaaataataatgaagctcatcttatacattttgctttcaccaattgattccttcttcttttagcccactattaaaacatttcttactgaatggttcatgtaggcttgctgaacagcacgcattacttgcttcctgaagagttcccccattcatccatttgtcccattagttgctgtggattatcaagttttgaaggaactgtacatcccaacagactgaaacattctaagtgaaatgagtataatccaagtaactggtgaactttggaggtttggagcttgaagagaatggctaagaagatttgaattatagggagggaacagaaatcatacatgaaaaggttttactgagaaggggaaaaccttagatagagggacatgtgaaacaaaatcatttgaaattttgattcagacatccatttccagtggcaaacagcaaagcctgaacccataaacccaaatgataggtgaagttgggtggttttatccaatgtctcaagcaagcaatgtctgggaatatcatagagtaacaagtgctggtcagccaaagaaacattcactgctggtgaaccaataccataagcatgtattatctaagcacttgatcaagaaatatacatgttgtacaagctctcaattttgttcatttattatcaaatttttaaaatacaagtttggtatgtgatttggaaaagatgccttctggatcttaagccagttgtcagtggaggtcctcagggctgcaaatgtcaagacataaccctgttcctcaccatcatgataccagatacaggtgaatacataggaactatctgcctgtgtcctcaatctcccttcaaacaagatgctgatttgtagggtacttggcaggttaaattaaaccagaagaggtgacttaataaaaaagggaatgacatttagggtataaagatctcataagaaatgtaatatgtaaattatatcttgctttatgttgtaaaatatacattgtttgcgctagaatagaaatgatttcttttcaataaaaagaaagaaggactctaaaaaaaaaaaaaaaa

SEQ ID NO: 17 - DNA, 2507 bp, Homo sapiens
  3284695CB1
cagagtgaaacttgtgcctggtgaccaaagtccctccaaagtgctcttccttctgggttattcaagccaaatatctgggtttccccctctcctcattccctagcaaaccccaattatcttccaagataggagatatttcccatccccttcctttgtaaatatctcatctcccactggagagcccaggagcctattcctggcatggatgttctgtccacacttgaggctgggcggtgtatcagacccttcaagcagcctggctggggcccaggactgagtctggggtcagctttcacggtcgcttttcccttcctcaccacccaccacagcccaccttgcatgcatggccagcccctccactccagcctgagccatgtgtgcccctgcgggaggacccattcatgccagaaagctggtaactccctcccagcatccctgcggaaggagtcagtttctgagagtgtgacttttcaaggcgaatgatggggaagggttccccagtccccacagtggccccacctctgggccctgcaccagagcccttctgtgtcacggcgggctgtgcacccatgcacacacctacgcacacacaacactccgcactgcagtatattcttgccaaagatttcctttaaaagcaagcacttttactaattattattttgtaaatgtttatcttcttctgtcttctccctccctgaatctattttactgttgtttattgttgaatctgtgtgtcagccaggagagcgctgtctggccttgaacatgggctgggatgggaaagggtctgggagaagatgggcaacaaagagccagggagtcatggacatcgcagcgacgcagaccccagcaggttcagtcccgtgctgccaccagctgtccagctgggtgtctggagggaagagggcagaggagggtcatgtcccttcagctgggggaggggcccagtgagctccacgtggctttttcccaaagggagcaagagggaaggattgggcgagaaaacaatggagaggggacctgcgaaggaaaacagggaggaagtgagcggtttgatcagcctgctatcacggtgttctggctctcttatttagccaggcgcttaagggacagatacatcacatcctaagtttgggaaaggcctttgacccatgtcatctgagcgtctcctccagtagctctgaaagctgtggacaccaatggccaggattccttctcccctggtttttgaggatccctgggtcttctgagactggccaggagagggatggtggggccagtggttgtgtgaaagcaggaggggcagccctcctggacaagtgtgatccccctataaacggctctcaggaggttagtgagtaggagattctgccttgttctgatgagcctgtgcaggggctccaggggagcatgctgtccagggggcacagaagggtggtgagtgtgatcaaatctagtctcactcccacttttttagtctcactcctacttttgtccaccacccctgcctcctggatcttctcccactttttttttcagctttaggacctggggagatcctgtgagtcaaggcagacacccaatcctgcccccacactcggggtcctccaagaggttggggggcagagtcccagagcagccctttaccccaggtccaggccctggaatcctgagactcgcgtttccttggccagtggtaacacaggacgtgtgtgcgcatgtgcaagtgtggatgtatgtgtgtgcgtgtgttttgctcatttctttagggaacttgggagtcggggttggaggtgctgggcaatggaacttcaaattcaatgtcgcccagcagtgaggggagtcgggaggtgaggcctgtaggccaaccaattggtggagtctcagcgatagcccaggtgagaagtggttcacccagaggggcagggtgggggcctcgggcagatctgtccctcttggcccctctgtcctcaaatgtccaaaatgttggaggacctctgttcatatcccacgcctgggctcttgccagcagtggagttactgtagagggatgtcccaagcttgttttccaatcagtgttaagctgtttgaaactctcctgtgtctgtgttttgtttgtgcgtgtgtgtgagagcacatcagtgtgtgcaggctgtgtttccccatttctctcctcccttcagacccatcattgagaacaaatgtaagaaatcccttcccaccaccctccctgcctcccaggccctctgcgggggaaacaagatcacccagcatccttccccaccccagctgtgtatttatatagatggaaatatactttatattttgtatcatcgtgcctatagccgctgccaccgtgtataaatcctggtgtatgctccttatcctggacatgaatgtattgtacactgacgcgtccccactcctgtacagctgctttgtttctttgcaatgcattgtatggctttataaatgataaagttaaagaaaaaaaaaaaaaaagg

SEQ ID NO: 18 - DNA, 2929 bp, Homo sapiens
  3481610CB1
aagctcggaattcggctcgagatgggttcctcatcccttcctgctgcaaaagaagttaacaaaaaacaagtgtgctacaaacacaatttcaatgcaagctcagtttcctggtgttcaaaaactgttgatgtgtgttgtcactttaccaatgctgctaataattcagtctggagcccatctatgaagctgaatctggttcctggggaaaacatcacatgccaggatcccgtaataggtgtcggagagccggggaaagtcatccagaagctatgccggttctcaaacgttcccagcagccctgagagtcccattggcgggaccatcacttacaaatgtgtaggctcccagtgggaggagaagagaaatgactgcatctctgccccaataaacagtctgctccagatggctaaggctttgatcaagagcccctctcaggatgagatgctccctacatacctgaaggatctttctattagcataggcaaagcggaacatgaaatcagctcttctcctgggagtctgggagccattattaacatccttgatctgctctcaacagttccaacccaagtaaattcagaaatgatgacgcacgtgctctctacggttaatatcatccttggcaagcccgtcttgaacacctggaaggttttacaacagcaatggaccaatcagagttcacagctactacattcagtggaaagattttcccaagcattacagtcaggagatagccctccattgtccttctcccaaactaatgtgcagatgagcagcatggtaatcaagtccagccacccagaaacctatcaacagaggtttgttttcccatactttgacctctggggcaatgtggtcattgacaagagctacctagaaaacttgcagtcggattcgtctattgtcaccatggctttcccaactctccaagccatccttgctcaggatatccaggaaaataactttgcagagagcttagtgatgacaaccactgtcagccacaatacgactatgccattcaggatttcaatgacttttaagaacaatagcccttcaggcggcgaaacgaagtgtgtcttctggaacttcaggcttgccaacaacacaggggggtgggacagcagtgggtgctatgttgaagaaggtgatggggacaatgtcacctgtatctgtgaccacctaacatcattctccatcctcatgtcccctgactccccagatcctagttctctcctgggaatactcctggatattatttcttatgttggggtgggcttttccatcttgagcttggcagcctgtctagttgtggaagctgtggtgtggaaatcggtgaccaagaatcggacttcttatatgcgccacacctgcatagtgaatatcgctgcctcccttctggtcgccaacacctggttcattgtggtcgctgccatccaggacaatcgctacatactctgcaagacagcctgtgtggctgccaccttcttcatccacttcttctacctcagcgtcttcttctggatgctgacactgggcctcatgctgttctatcgcctggttttcattctgcatgaaacaagcaggtccactcagaaagccattgccttctgtcttggctatggctgcccacttgccatctcggtcatcacgctgggagccacccagccccgggaagtctatacgaggaagaatgtctgttggctcaactgggaggacaccaaggccctgctggctttcgccatcccagcactgatcattgtggtggtgaacataaccatcactattgtggtcatcaccaagatcctgaggccttccattggagacaagccatgcaagcaggagaagagcagcctgtttcagatcagcaagagcattggggtcctcacaccactcttgggcctcacttggggttttggtctcaccactgtgttcccagggaccaaccttgtgttccatatcatatttgccatcctcaatgtcttccagggattattcattttactctttggatgcctctgggatctgaaggtacaggaagctttgctgaataagttttcattgtcgagatggtcttcacagcactcaaagtcaacatccctgggttcatccacacctgtgttttctatgagttctccaatatcaaggagatttaacaatttgtttggtaaaacaggaacgtataatgtttccaccccagaagcaaccagctcatccctggaaaactcatccagtgcttcttcgttgctcaactaagaacaggataatccaacctacgtgacctcccggggacagtggctgtgcttttaaaaagagatgcttgcaaagcaatggggaacgtgttctcggggcaggtttccgggagcagatgccaaaaagactttttcatagagaagaggctttcttttgtaaagacagaataaaaataattgttatgtttctgtttgttccctccccctcccccttgtgtgataccacatgtgtatagtatttaagtgaaactcaagccctcaaggcccaacttctctgtctatattgtaatatagaatttcgaagagacattttcactttttacacattgggcacaaagataagctttgattaaagtagtaagtaaaaggctacctaggaaatacttcagtgaattctaagaaggaaggaaggaagaaaggaaggaaagaagggagggaaacagggagaaagggaaaaagaagaaaaagagatagatgataataggaacaaataaagacaaacaacattaaggggcatattgtaagatttccatgttaatgatctaatataatcactcagtgccacattttgagaatttttttttttaatgggcttcaaaaattggaaaactgtgaaagctaagtccattggggggaatggaattacttttgggggccagtatctttcctttgattgttcc

SEQ ID NO: 19 - DNA, 1725 bp, Homo sapiens
  3722004CB1
gaggcaagaattcggcacgagggagagcccgcgggcgtgggggagctcggggacctgcggaccgggggagcccgaacgagggggatcccgcggcggcgccagcgaggcggaggagcaggcggtggaggcgaggcaggaagaggagcaggacttggatggtgagaaggggccatcatcggaagggcctgaggaggaggacggagaaggcttctccttcaaatacagccccgggaagctgaggggaaaccagtacaagaagatgatgaccaaagaggagctggaggaggagcagaggattgagctgacctctgacctcacttccctgtagcaagttccttaggtcctgagccacaaatattcttgcaaatccttttgaactgaagaataacgaagttatccttagcgtcctcctaaaggcttttccttttggcatcttaaaagcttgagagataaaacggaaaccccagagaggagtctgggcaggctcccagggtgcatgctgcctccataaatctgctgagctctagaccctcaatcaggacttgtcccttggctagcaggatcctgggaacacctttggccctgccctgtgtagagatgttcatgtctgttcctgtgggtcactttgttaagctgaagagttttaagaggtagagctcagaccctggactgggatttttcttaccactcaaacttgctatccacacaccctgcacaccttagataaaaagaacattttaaaagcagagttcactttcactccagtctcccctcttttgccctcactgaagccaaaccacagaagactttgaggaatgagagacaaatgaggtagagctcacctgtgctcaccagctccgtcagggtggtcagccgacccctttccctgggaaccccacttctctctgtggctggcttggttgtcgggggtgagatgccatattgattacagggcagcaaagaaccagtaccaggaatttacttgaccattccccttatttttcatctagaggaatctcggattcagccctttcattgctaagacaccttttcactgaggttcttaccagctcagccaaatctccactctgctatagcagaagcaataatgtttgctttaaaaagatttcttgacctatgccttttcttagaaagtttgatagattagttagaacttcagatcatcagatcagtctcaaatgggtttcttggaattttatatttgacaatatttatactataccaaactcatttgcagttcttaggtttgttggttaaaacatttttttaaagcagtaagtttatagaaaatgttttcatttaatggaaggctggggaatgtccagcatcaacccctatggcatgcattcccagtggccttctcatctgggcctggaacctttggttcagggcttaggggagaacaggccacatggcaacagccacacagtcattgccttcaacacagagccacgtgtccccaaacagcaatagtcatgcccttgtccaggctgggatctaattgatacaataggtcgttgactccctcctagtagagctatctaggtttgtctggaaagtttccgaccctggcttataggcaccacacctcatgtactcctcatggcttggatctctgtattcagcctttgttcagtccaataaactttgagtagatgatctcaaaaaaaaaaaaaaaaaggccggcgcaagcttattcctttt

SEQ ID NO: 20 - DNA, 1987 bp, Homo sapiens
  3948614CB1
gacggccagtgcaagctaaaattaaccctcactaaagggaataagcttgcggccgcctggagctctcggcctcggcttcgacgacggcaacttctcgctgctcatccgcgcggtggaggagacggacgcggggctgtacacctgcaacctgcaccatcactactgccacctctacgagagcctggccgtccgcctggaggtcaccgacggccccccggccacccccgcctactgggacggcgagaaggaggtgctggcggtggcgcgcggcgcacccgcgcttctgacctgcgtgaaccgcgggcacgtgtggaccgaccggcacgtggaggaggctcaacaggtggtgcactgggaccggcagccgcccggggtcccgcacgaccgcgcggaccgcctgctggacctctacgcgtcgggcgagcgccgcgcctacgggcccctttttctgcgcgaccgcgtggctgtgggcgcggatgcctttgagcgcggtgacttctcactgcgtatcgagccgctggaggtcgccgacgagggcacctactcctgccacctgcaccaccattactgtggcctgcacgaacgccgcgtcttccacctgacggtcgccgaaccccacgcggagccgcccccccggggctctccgggcaacggctccagccacagcggcgccccaggcccagaccccacactggcgcgcggccacaacgtcatcaatgtcatcgtccccgagagccgagcccacttcttccagcagctgggctacgtgctggccacgctgctgctcttcatcctgctactggtcactgtcctcctggccgcccgcaggcgccgcggaggctacgaatactcggaccagaagtcgggaaagtcaaaggggaaggatgttaacttggcggagttcgctgtggctgcaggggaccagatgctttacaggagtgaggacatccagctagattacaaaaacaacatcctgaaggagagggcggagctggcccacagccccctgcctgccaagtacatcgacctagacaaagggttccggaaggagaactgcaaatagggaggccctgggctcctggctgggccagcagctgcacctctcctgtctgtgctcctcggggcatctcctgatgctccggggctcaccccccttccagcggctggtcccgctttcctggaatttggcctgggcgtatgcagaggccgcctccacacccctcccccaggggcttggtggcagcatagcccccacccctgcggcctttgctcacgggtggccctgcccacccctggcacaaccaaaatcccactgatgcccatcatgccctcagacccttctgggctctgcccgctgggggcctgaagacattcctggaggacactcccatcagaacctggcagccccaaaactggggtcagcctcagggcaggagtcccactcctccagggctctgctcgtccggggctgggagatgttcctggaggaggacactcccatcagaacttggcagccttgaagttggggtcagcctcggcaggagtcccactcctcctggggtgctgcctgccaccaagagctcccccacctgtaccaccatgtgggactccaggcaccatctgttctccccagggacctgctgacttgaatgccagcccttgctcctctgtgttgctttgggccacctggggctgcaccccctgccctttctctgccccatccctaccctagccttgctctcagccaccttgatagtcactgggctccctgtgacttctgaccctgacacccctcccttggactctgcctgggctggagtctagggctggggctacatttggcttctgtactggctgaggacaggggagggagtgaagttggtttggggtggcctgtgttgccactctcagcaccccacatttgcatctgctggtggacctgccaccatcacaataaagtccccatctgatttttaaaaaaaaaaaa

SEQ ID NO: 21 - protein, 551 aa, Homo sapiens
  627722CD1
MEEAELVKGRLQAITDKRKIQEEISQKRLKIEEDKLKHQHLKKKALREKWLLDGISSGKEQEEMKKQNQQDQHQIQVLEQSILRLEKEIQDLEKAELQISTKEEAILKKLKSIERTTEDIIRSVKVEREERAEESIEDIYANIPDLPKSYIPSRLRKEINEEKEDDEQNRKALYAMEIKVEKDLKTGESTVLSSIPLPSDDFKGTGIKVYDDGQKSVYAVSSNHSAAYNGTDGLAPVEVEELLRQASERNSKSPTEYHEPVYANPFYRPTTPQRETVTPGPNFQERIKIKTNGLGIGVNESIHNMGNGLSEERGNNFNHISPIPPVPHPRSVIQQAEEKLHTPQKRLMTPWEESNVMQDKDAPSPKPRLSPRETIFGKSEHQNSSPTCQEDEEDVRYNIVHSLPPDINDTEPVTMIFMGYQQAEDSEEDKKFLTGYDGIIHAELVVIDDEEEEDEGEAEKPSYHPIAPHSQVYQPAKPTPLPRKRSEASPHENTNHKSPHKNSISLKEQEESLGSPVHHSPFDAQTTGDGTEDPSLTALRMRMAKLGKKVI

SEQ ID NO: 22 - protein, 99 aa, Homo sapiens
  1556751CD1
MEALANVNFPRKSFRPEDAGKESGSQGGFCVPAARPQTMVTGPSCSSPGLQNFSPQRKENRACACWQNAGPAPKNPMCVRLKVGRPQASQRKLKETGLC

SEQ ID NO: 23 - protein, 493 aa, Homo sapiens
  2268890CD1
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH